(19) United States
(12) Patent Application Publication
(10) Pub. No.: US 2010/0174129 A1
(43) Pub. Date: Jul. 8, 2010

(54) HIGH THROUGHPUT PROPYLENE FROM METHANOL CATALYTIC PROCESS DEVELOPMENT METHOD

(76) Inventors: Richard F. Bauman, Bellingham, WA (US); Rocco A. Fiato, Basking Ridge, NJ (US)

Correspondence Address: Jamie Zheng, 3524 Waverley Street, Palo Alto, CA, 94306, US

(21) Appl. No.: 12/521,579

(22) PCT Filed: Dec. 29, 2007
(86) PCT No.: PCT/CN07/71393

§ 371 Date: Jun. 29, 2009

Related U.S. Application Data
(60) Provisional application No. 60/882,699, filed on Dec. 29, 2006.
(60) Provisional application No. 60/882,812, filed on Dec. 29, 2006.

(51) Int. Cl. C07C 1/20

(52) U.S. Cl. 585/501

(57) ABSTRACT

A catalytic process development apparatus and method for simulating a commercial scale methanol and/or DME to propylene catalytic conversion system that includes a plurality of series-connected plug-flow reactors. The method involves simulating the operation of the series-connected plug-flow reactors by operating a series of multistage series-connected laboratory scale plug-flow reactors, the stages of which each containing a zeolite catalyst bed, each of the laboratory scale reactors corresponding to a separate one of the commercial scale series-connected reactors. Fresh feed, including methanol and/or DME, is supplied to the first of the laboratory scale reactor stages, and selected ones of steam, methanol and/or DME, contaminants and reaction products are supplied to selected ones of the laboratory scale reactor stages. The simulation is repeated at different sets of operating conditions and catalyst characteristics.

### FIELD OF INVENTION

This invention relates to methods for the low cost, accelerated development of methanol and/or dimethyl ether (“DME”) to propylene (DTP) catalysts and corresponding fixed bed catalytic processes from discovery to commercial readiness.

### BACKGROUND OF THE INVENTION

In order to scale-up a fixed bed methanol (or DME) to propylene (MTP or DTP) catalytic process, it is necessary to define the impact of time on stream, residence time, catalyst particle size, shape and other characteristics, and temperature profile on reaction rate and selectivity, and deactivation rate of the catalyst.

The first step in a traditional scale-up program generally involves the selection and definition of the intrinsic properties of the catalyst. This step is typically performed isothermally with a diluted, crushed or powdered catalyst to minimize mass transfer limitations. A process variable study is performed to determine the impact of space velocity, pressure, and residence time on reaction rate and selectivity. Activity and selectivity maintenance are then determined over a six to twelve month operating period. At the end of the operation, a second process variable study is performed to determine whether these properties have changed during time on stream. Next, a commercial form of the catalyst is tested in an isothermal reactor. The commercial catalyst is of a larger particle size than the crushed catalyst and may have a special shape to minimize pressure drop during operation. The larger particle size generally results in a lower reaction rate and a selectivity loss due to limitations on mass transfer of reactants or products in and out of the catalyst pores. Operations generally consist of performing process variable studies at the beginning and end of an activity and selectivity maintenance run. This operation can be run in a laboratory scale reactor and typically lasts approximately one year.

The final step in the scale-up process is to test the commercial catalyst under adiabatic conditions, normally in a demonstration scale reactor containing one or more reactor tubes. The tubes in the demonstration scale reactor would have internal diameter of approximately 1 inch. In some cases, to further explore heat transfer effects, a configuration containing up to about 6-8 tubes arranged at commercial spacing could be used. In an exothermic reaction, the temperature profile depends upon the degree to which heat is continuously removed, as in a tubular reactor, or the reactor is simply a fixed bed reactor without a specific heat removal capability. The temperature profile can have a significant impact on selectivity, reaction rate, and activity maintenance. The test run also provides a measure of the tendency for the catalyst to produce hot spots or temperature runaways. Here again, the operating period can exceed one year.

This sequential approach typically takes in excess of three years to complete and may not provide all of desired data. For many catalysts, the reaction rate and selectivity may be a function of residence time as well as time on stream. This can be the result of changes in the catalyst state or form, due to exposure for extended periods of time, or it may be due to the changing gas and liquid composition from the reactor inlet to the outlet. Examples would include oxidation from water formed during conversion, formation of a support over layer, poisoning, e.g., by reaction with hydrogen sulfide and ammonia, etc. In addition, surface catalytic reactions and buildup of feed and products in the pores can result in reductions in mass transfer to the catalyst.

More recently, High Throughput Experimentation (HTE) techniques have been proposed as a source of data for new catalysts and processes. These HTE experiments are normally performed under conditions that minimize heat and mass transfer effects. Small volumes (less than 2 ml) of catalyst and high heat transfer rates are utilized. This approach is useful for comparing the intrinsic properties of an array of candidate catalysts but does not provide the data required for scale-up. See, for example, U.S. Pat. Nos. 6,149,882 and 6,869,799.

In addition, there are several studies where high throughput experimentation has been proposed for use in the development of multi-channel reactors, see for example U.S. Pat. No. 6,806,087, and for optimization of Co—Ru FT catalysts, see for example U.S. Pat. No. 6,649,662.

A large portion of the worldwide petrochemical industry is concerned with the production of light olefin materials and their subsequent use in the production of numerous important chemical products via polymerization, oligomerization, alkylation and similar well-known chemical reactions. Light olefins include ethylene, propylene and mixtures thereof. These light olefins are essential building blocks for the modern petrochemical and chemical industries. The major source for these materials in present day refining is the steam cracking of petroleum feeds. For various reasons including geographical, economic, political and diminished supply considerations, a source other than petroleum has long been sought for the massive quantities of raw materials that are needed to supply the demand for these light olefin materials. A great deal of the prior art's attention has been focused on the possibility of using hydrocarbon oxygenates, and particularly methanol, as a prime source of the necessary alternative feedstock. Oxygenates are particularly attractive because they can be produced from such widely available materials as coal, natural gas, recycled plastics, various carbon waste streams from industry and various products and by-products from the agricultural industry. The art of making methanol from these types of raw materials is well established.

Essentially two major techniques have been discussed for conversion of methanol to light olefins (“MTO”). The first of these MTO processes is based on early German and American work with a catalytically conversion zone containing a zeolitic type of catalyst system. Representative of the early German work is U.S. Pat. No. 4,387,263. This '263 patent discloses a series of experiments with methanol conversion techniques using a ZSM-5-type of catalyst system wherein the problem of DME recycle is a major focus of the technology. Although good yields of ethylene and propylene were reported in this '263 patent, they unfortunately were accompanied by substantial formation of higher aliphatic and aromatic hydrocarbons, which the patentees speculated might be useful as an engine fuel and specifically as a gasoline-type of material. In order to limit the amount of this heavier material that is produced, the patentees of the '263 patent propose to limit conversion to less than 80% of the methanol charged to the MTO conversion step. This operation at lower conversion levels necessitated the recovering and recycling not only unreacted methanol but also substantial amounts of a DME intermediate product. The focus of the '263 patent invention was therefore on a DME and methanol scrubbing step utilizing a water solvent in order to efficiently recapture the light olefin value of the unreacted methanol and of the intermediate reactant DME.

U.S. Pat. No. 4,587,373 recognized that a commercial plant would have to operate at a pressure substantially above the preferred range disclosed in the '263 patent in order to make commercial equipment of reasonable size possible with commercial mass flow rates. The '373 patent recognized that the higher pressure zeolitic MTO route results in a substantial additional loss of DME caused by dissolution of substantial quantities of DME in the heavy hydrocarbon oil by-product recovered from the liquid hydrocarbon stream withdrawn from the primary separator.

Because of an inability of this zeolitic MTO route to control the amounts of undesired C₄⁺ hydrocarbon products produced by the ZSM-5 catalyst system, a second MTO conversion technology was developed based on the use of a non-zeolitic molecular sieve catalytic material. See e.g., U.S. Pat. Nos. 5,095,163, 5,126,308 and 5,191,141. This second approach to MTO conversion technology was primarily based on using a catalyst system comprising a silicoaluminophosphate molecular sieve (SAPO) and especially SAPO-34. This SAPO-34 material was found to have a number of advantages, including very high selectivity for light olefins with a methanol feedstock and consequently very low selectivities for the undesired corresponding light paraffins and the heavier materials.

However, the problem of DME co-production is also present in the SAPO process discussed above. In U.S. Pat. No. 4,382,263, a relatively high pressure DME absorption zone is taught utilizing a plain water solvent in order to recapture and recycle the DME intermediate. U.S. Pat. No. 4,587,373 focused on utilizing a more efficient DME solvent in the DME absorption zone and recommended that a portion of the methanolic feed to the MTO conversion reactor be diverted to the DME absorption zone in order to more efficiently recapture the DME contaminant from the olefin product stream.

It has been found however, that if a portion of the methanol feed to the MTO conversion zone is diverted to the DME absorber as suggested in the '373 patent in order to recover DME more efficiently, there is substantial co-absorption of light olefins into the methanol solvent associated with this scheme. This greatly complicates the design of an efficient product work-up flow scheme for a SAPO based MTO conversion zone. For example, when the DME absorption zone is operated with a methanol solvent at scrubbing conditions including a temperature of about 54° C. (129° F.) and a pressure of about 2020 kPa (293 psi) with a 99.85 mass-% methanol solvent, at least 12.3 mass-% of the C₂ olefins and 40.3 mass-% of the C₃ olefins charged to the DME scrubber are co-absorbed in the DME-rich liquid solvent bottom stream withdrawn from the scrubber. When this DME-rich solvent stream is recycled to the MTO conversion zone, a substantial internal circuit of light olefins is created which acts to substantially increase the size of the MTO conversion zone and the rate of detrimental coking on the catalyst contained therein due to the fact that these C₂ and C₃ olefins are reactive and can undergo polymerization and condensation to form coke precursors.

In typical operations in the above-mentioned process of the '373 patent, and in related processes of U.S. Pat. No. 5,602,289 and U.S. Pat. No. 4,404,414, using a Zeolite catalyst, the Zeolite catalyst is disposed in a single uniform bed or in a plurality of series-connected packed bed reactors, each containing the same Zeolite catalyst. The methanol is first converted in part to produce a mixture of methanol, DME and H₂O, typically using an Al₂O₃ dehydration catalyst that usually is contained as a bed in a packed bed reactor. Details of a suitable Al₂O₃ catalyst are disclosed in EP 0 448 000 B1 and DE 197 23 363 A1.

In the series-connected reactor embodiment, such as described in the '414 patent, a first partial stream of the partially converted methanol output of the Al₂O₃ containing reactor, consisting of a vapor mixture of methanol, DME, H₂O and, optionally, additional steam is introduced into the first Zeolite catalyst containing series-connected reactor, a first intermediate product mixture is withdrawn from the outlet of such first reactor and is charged into the second series-connected packed bed reactor. A second partial stream of the first vapor mixture is also supplied to the second series-connected packed bed reactor. Product mixture is withdrawn from the last one of the series-connected packed bed reactors and cooled. A fraction of such product mixture rich in propane is separated and residual substances are obtained, which are in part gaseous and contain C₃⁺ hydrocarbons. At least part of the residual substances are recirculated into at least one of the packed bed reactors. Usually, the zeolite catalyst is disposed as beds in a maximum of four or five series-connected packed bed reactors. The separation of the fraction rich in propane may be effected in a manner known per se, for instance by distillation or by adsorption.

The Zeolite catalysts used in the conversion of methanol and/or DME to propylene become progressively deactivated during use. A major portion of such deactivation results from the formation of a carbon overlayer on the catalyst surface. It is therefore necessary periodically to regenerate the catalyst by removing the carbon overlayer to the extent possible. This is generally accomplished by treating the catalyst beds with high temperature hydrogen, or combination of oxygen and an inert gas. Regeneration of the zeolite catalyst with a hydrogen containing stream is performed at temperatures typically in the range of 200 to 600 degree C., while regeneration with oxygen, such as described in U.S. Pat. No. 4,795,845, is performed at higher temperatures typically in the range of 300 to 700 degree C. Aged methanol/methyl ether conversion catalysts can be regenerated in conventional manner by contacting the catalyst at elevated temperature with an oxygen-containing gas such as air to effect controlled burning of coke from the deactivated catalyst. While such a conventional regeneration procedure can restore catalytic activity diminished by coke formation on the catalyst during methanol/methyl ether conversion, regeneration in this manner must be conducted in the absence of organic reactants and preferably in a separate regeneration zone which is remote from the methanol/methyl ether conversion zone. Furthermore, catalyst regeneration by controlled burning of coke produces water and carbon dioxide, and water at high temperatures can permanently destroy the structure of the zeolite catalyst and thus can actually diminish catalytic activity in some instances. There is, therefore, a continuing need to develop additional catalyst regeneration procedures which can be employed to restore the diminished activity of the zeolite-based catalysts which have been used to promote the conversion of methanol and/or methyl ether to hydrocarbon products selectively enriched in light olefins.

There is a continuing need to develop better methods for efficient operation and regeneration of deactivated methanol/DME conversion catalysts and to devise methods that allow such materials to be developed in an efficient and cost effective manner.

### SUMMARY OF THE INVENTION

This invention relates to a low cost, accelerated method for determining an advantageous combination of reactor structures, catalyst characteristics, catalyst bed structures and process conditions for scaling up from discovery to commercial readiness a plug-flow catalytic process for producing propylene from methanol (and/or DME) having high propylene productivity and selectivity and minimizing production of heavy (C₅⁺) hydrocarbons, catalyst poisoning and deactivation, and in which the catalyst can be efficiently regenerated. The plug-flow catalytic process generally involves reacting methanol vapor on a first catalyst to obtain a first vapor mixture containing DME, and wherein a product mixture containing propylene is produced from the DME containing vapor mixture in a set of series-connected plug-flow reactors having catalyst beds preferably containing zeolite-containing catalysts.

The method of the invention involves the use of high throughput laboratory scale catalytic process development apparatus that includes one or more composite multistage series-connected laboratory scale plug-flow reactors for simulating a set of series-connected plug-flow reactors, wherein separate series-connected pluralities of the stages of the laboratory scale reactor correspond to separate ones of the series-connected plug-flow reactors. The method further involves performing successive simulation steps involving successively testing of one or more catalysts in one or more forms in a plurality of catalyst bed configurations in the stages of the multistage laboratory reactors under a plurality of sets of process conditions. The characteristics and compositions of the effluents of the laboratory scale reactor stages are measured during each simulation step, and the results of such measurements are used to help determine the choice of catalyst bed characteristics and process conditions in subsequent simulation steps for improving the productivity and selectivity of the conversion of methanol and/or DME to propylene.

The process conditions under which the testing is performed include various sets of temperatures, pressures, flow rates and relative partial pressures of methanol, DME, H₂O and reaction products in the catalyst beds of the various stages of the laboratory reactors. The testing of the catalysts includes determining the effects, in the various sets of process conditions, of catalyst activity, surface acidity and dopants on the performance and deactivation of the catalysts in various portions of the series-connected fixed bed reactors over time. The testing of catalyst bed configurations can include the testing of the effects of various configurations of catalyst beds in the laboratory scale reactor stages, including varying the catalyst characteristics from one laboratory scale reactor stage to the next.

Deactivating carbon layers deposited on the surface of zeolite containing catalysts may range from a carbon, which is relatively loosely bound to the catalyst surface and can be easily removed during regeneration, to δ carbon, which is very tightly bound to the catalyst surface and requires a much more severe treatment during regeneration in order to remove it. The testing of zeolite containing catalysts in accordance with the invention includes determining the amounts and types of carbon deposited on the catalyst surface in the different laboratory scale reactor stages under various different operating conditions, the regeneration conditions required to remove such carbon overlayers from the catalyst surface in each of the reactor stages, and investigating the trade-offs between treating the catalysts with hydrogen or oxygen under temperature conditions severe enough to provide adequate regeneration, while not so severe to cause an unacceptable degree of sintering of the catalyst. The method of the invention further includes the investigation of the effects on the catalysts of other deactivating species that may be present in the reactant feeds or products, such as ammonia, aromatics, carbon monoxide and hydrogen sulfide, and the investigation of methods of regenerating catalysts that have been deactivated in some measure by such species.

The data generated as a result of this testing enables the design of a methanol and/or DME to propylene catalytic system in which the catalyst characteristics, partial pressures of methanol, DME and steam feeds, temperatures and flow rates are optimized for the productivity and selectivity of propylene and catalyst life. The generated data will also define optimal regeneration conditions for regenerating the catalyst in the different longitudinal portions of the catalyst beds in the commercial scale series-connected reactors.

The term “plug-flow reactor”, as used herein refers to fixed bed reactors, packed bed reactors, trickle bed reactors and monolithic reactors operating either in a once through or a recycle mode. The term “laboratory scale plug-flow reactor” as used herein, refers to a plug-flow reactor in which each reactor stage has an internal diameter of less than 4 inches, preferably less than 2 inches, and more preferably less than 1 inch; a length of less than 8 feet, preferably less than 4 feet, more preferably less than 1 foot; and a catalyst charge of less than 800 grams, preferably less than 400 grams, more preferably less than 25 grams (excluding inert diluent particles charged to the reactor).

### BRIEF DESCRIPTION OF THE DRAWINGS

FIG. 1 is a schematic representation of a composite multistage, series-connected, fixed bed reactor in accordance with the invention;

FIG. 2 is a schematic representation of a composite multistage, series-connected, fixed bed reactor and a parallel multistage, series-connected, probe reactor in accordance with the invention;

FIG. 3 is a schematic representation of a composite multistage, series-connected, fixed bed reactor and a fluid dynamically linked, single stage probe reactor in accordance with another embodiment of the invention;

FIG. 4 is a schematic representation of a composite multistage, series-connected, fixed bed reactor and a fluid dynamically linked, multistage, series-connected, probe reactor in accordance with the invention;

FIG. 5 is a schematic representation of a multistage, composite series-connected, fixed bed reactor disposed in a constant temperature environment in the form of a fluidized sand bath in accordance the invention;

FIG. 6 is a schematic representation of a plurality of composite multistage, series-connected, fixed bed reactors disposed in the common fluidized sand bath in accordance with the invention;

FIG. 7 is a schematic representation of a plurality of composite multistage, series-connected, fixed bed reactors configured to receive controlled variable inputs in accordance with the invention;

FIG. 8 is a graph useful for determining the Thiele Modulus of a catalyst;

FIG. 9 is a graph of the Effectiveness Factor versus Thiele Modulus for a catalyst;

FIG. 10 is a schematic representation of a plug-flow reactor arrangement in accordance with another embodiment of the invention;

FIG. 11 is a typical chromatograph of the reactor down-stream components of MTP/DTP Products;

FIG. 12 is a schematic representation of a multistage, composite series-connected, isothermal plug flow reactor in accordance with the invention;

FIG. 13 illustrates an assembled, schematic diagram of reactors and a separator in accordance with one embodiment of the present invention;

FIG. 14 illustrates an assembled, schematic diagram of the reactors and the separator in accordance with another embodiment of the present invention; and

FIG. 15 illustrates an assembled, schematic diagram of the reactor and the separator in accordance with yet another embodiment of the present invention.

### DESCRIPTION OF THE PREFERRED EMBODIMENTS

In the conversion of methanol and/or DME to propylene, it is desirable to the extent practicable to maximize the productivity and selectivity to propylene and the ratio of propylene to ethylene, and to minimize the production of higher molecular weight (C₅⁺) hydrocarbons. It is also important to minimize to the extent practicable the deactivation of the zeolite containing catalysts in the series-connected plug-flow reactors used in the process. An important component of this deactivation results from the carbon overlayer formation on the catalyst surface. The degree of difficulty involved in regenerating the zeolite containing catalysts by removing the carbon overlayer is also an important consideration in the commercial scale catalytic conversion system design.

The inventors have found that, in the conversion of methanol and/or DME to propylene in a plug-flow reactor system that includes a plurality of series-connected plug-flow reactors having catalyst beds preferably containing zeolite catalysts, the increasing partial pressure of the olefin products in the reactant vapor mixture being supplied to the successive plug-flow reactor stages can result in the product olefins reacting in the presence of the zeolite catalyst to form undesirable higher molecular weight hydrocarbons and deactivating carbon layers on the catalyst surface. Such reactions not only decrease the productivity and selectivity to the desired propylene product, but also significantly shorten the catalyst life. By controlling the acidity and strength of the catalysts in the catalyst beds of the successive commercial scale series-connected plug-flow reactors, and to vary this property in a controlled way longitudinally within the individual catalyst beds, it is possible, in accordance with the invention, to minimize these undesirable reactions, to maximize the productivity and selectivity to propylene of the catalyst system, and to extend the catalyst life.

An optimal set of reactant and product partial pressures, flow rates, temperatures, catalyst beds structures and longitudinal variation in catalyst activity levels in the catalyst beds of a set of series-connected plug-flow reactors in a methanol and/or DME to propylene catalytic conversion system depends upon the particular type and characteristics of the catalyst or catalysts being used and the physical characteristics of the series-connected reactors, and may be different for different conversion system configurations. The method of the invention investigates this multivariable space to generate data for a favorable set of operating conditions and trade-offs for a commercial scale reactor system having high productivity and selectivity of propylene with good catalyst life and catalyst regeneration characteristics.

As illustrated by the data in the following tables, it has been determined that various treatments, additives and operating conditions can increase the productivity and selectivity to propylene of zeolite containing catalysts in plug-flow reactors. Tested reaction temperatures were 370, 420, and 470° C. Five commercially a available catalysts were chosen to undergo this test (catalyst loading amount 0.5 g, diluted to 3 ml, WHSV=2 h⁻¹, the reactants were 80 wt % methanol and 20 wt % H₂O). The product distributions at these temperatures are profoundly different as shown in Table 1.

TABLE 1
Product distribution at various temperatures

| Catalyst (Si/Al) | T(° C.) | CH₄ | C₂H₄ | C₂H₆ | C₃H₆ | C₃H₈ | C₄ | C₅+ | C₃⁼/C₂⁼ |
|---|---|---|---|---|---|---|---|---|---|
| HZSM-5 (25) | 370 | 0.5 | 0.5 | 0.5 | 0.8 | 15.2 | 18.3 | 64.2 | 1.6 |
| | 420 | 2.6 | 2.1 | 0.7 | 3.4 | 12.8 | 19.0 | 59.5 | 1.6 |
| | 470 | 6.4 | 7.2 | 0.9 | 10.2 | 7.0 | 15.8 | 52.6 | 1.4 |
| HZSM-5 (38) | 370 | 0.5 | 0.5 | 0.5 | 0.9 | 15.2 | 18.8 | 63.6 | 1.8 |
| | 420 | 2.5 | 2.3 | 0.7 | 3.6 | 14.2 | 19.1 | 57.7 | 1.6 |
| | 470 | 6.5 | 7.9 | 0.8 | 10.7 | 6.8 | 15.6 | 51.7 | 1.4 |
| HZSM-5 (62) | 370 | 0.4 | 0.7 | 0.3 | 1.3 | 10.7 | 18.1 | 68.5 | 1.9 |
| | 420 | 2.1 | 2.0 | 0.7 | 3.3 | 12.7 | 18.5 | 60.8 | 1.7 |
| | 470 | 7.4 | 6.7 | 0.9 | 9.6 | 7.7 | 14.7 | 53.1 | 1.4 |
| HZSM-5 (99) | 370 | 0.4 | 2.5 | 0.2 | 5.0 | 6.0 | 19.2 | 66.8 | 2.0 |
| | 420 | 1.6 | 5.3 | 0.4 | 11.1 | 7.1 | 21.3 | 53.1 | 2.1 |
| | 470 | 5.5 | 11.0 | 0.6 | 21.7 | 4.2 | 18.8 | 38.4 | 2.0 |
| HZSM-5 (360) | 370 | 0.3 | 7.7 | 0.2 | 9.4 | 2.5 | 19.4 | 60.4 | 1.2 |
| | 420 | 0.8 | 6.4 | 0.3 | 17.4 | 3.2 | 24.4 | 47.6 | 2.7 |
| | 470 | 5.0 | 9.6 | 0.4 | 25.0 | 2.2 | 20.5 | 37.3 | 2.6 |

(catalyst loading amount 0.5 g, diluted to 3 ml, WHSV = 2 h⁻¹)

The 5 selected catalysts were proton form HZSM-5 from different vendors and having the different Si/Al ratios indicated in parenthesis. In all instances, the methanol conversion was 100%. At lower reaction temperatures, main products over all catalysts were C5⁺ gasoline distillates, butane and propane. Propylene and ethylene were only produced at very low selectivity and low C3⁼/C2⁼ ratios. MTG is the dominating reaction. At higher temperatures, the selectivity towards C3⁼ increases, i.e., MTO and MTP become more significant at higher temperatures. We thus select 470° C. as the reaction temperature for further screening experiments. Furthermore, an important conclusion may be drawn from the data in Table 1. Under the tested conditions, the MTP selectivity significantly depends on the Si/Al ratio of the materials. At high Si/Al, not only does propylene yields increase, the C3⁼/C2⁼ ratio increase as well.

Two experiments testing the effects of WHSV have been carried out, one in a 16 channel parallel reactor system, the other one using the single fixed-bed reactor. In the parallel reactor experiment, one catalyst, the proton form HZSM-5 with Si/Al=62 was loaded into 16 reactor channels with various amounts, in order to achieve WHSV=2, 5 and 10 h⁻¹, respectively. At the stable states, the methanol conversion in all reactor channels was 100%, and the products distributions at different WHSV are listed in Table 2.

TABLE 2
Product distribution at different WHSV.

| WHSV | CH₄ | C₂H₄ | C₂H₆ | C₃H₆ | C₃H₈ | C₄ | C₅+ | C₃⁼/C₂⁼ |
|---|---|---|---|---|---|---|---|---|
| 2 | 7.4 | 6.7 | 6.9 | 9.6 | 7.7 | 14.7 | 53.1 | 1.4 |
| 5 | 6.3 | 13.7 | 0.4 | 18.3 | 2.4 | 17.5 | 42.4 | 1.3 |
| 10 | 4.2 | 12.8 | 0.4 | 17.4 | 4.3 | 19.5 | 41.5 | 1.4 |

Catalyst HZSM-5(62), T = 470° C.

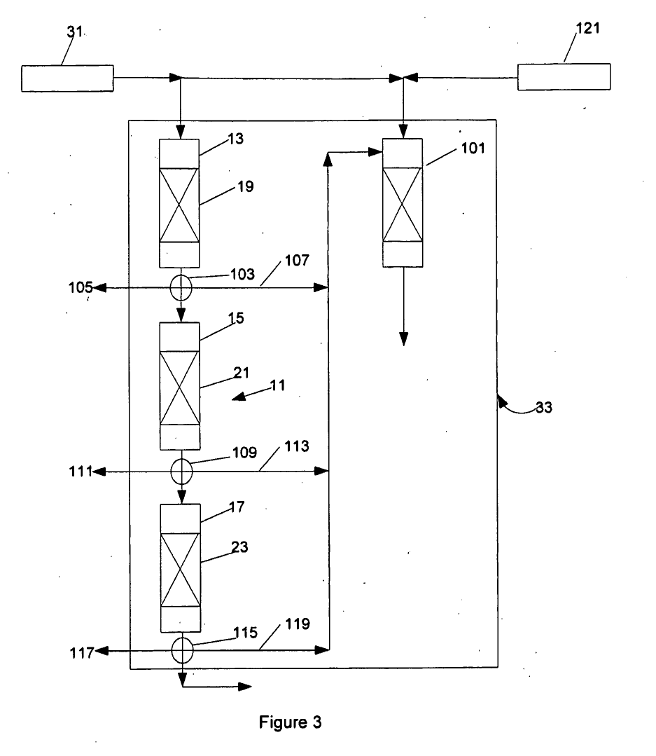

The single bed reactor experiment used a Mg²⁺ exchanged MgZSM-5(260), with Si/Al=260. Again the reaction temperature was 470° C. However, because of the different inner diameter of the reactors, the contact time is different from that for the parallel reactor experiment. Tested WHSV in this experiment were 2, 4, 8, 12, 16, 20, 24 h⁻¹. The results are shown in FIG. 3.

TABLE 3
Product distribution at different WHSV.

| WHSV | CH₄ | C₂H₄ | C₂H₆ | C₃H₆ | C₃H₈ | C₄ | C₅+ | C₃⁼/C₂⁼ |
|---|---|---|---|---|---|---|---|---|
| 2 | 0.9 | 9.0 | 0.1 | 32.4 | 1.6 | 28.1 | 27.9 | 3.6 |
| 4 | 0.6 | 10.3 | 0.1 | 34.3 | 1.7 | 26.0 | 27.1 | 3.4 |
| 8 | 0.9 | 7.1 | 0.1 | 36.5 | 0.8 | 28.5 | 26.1 | 5.2 |
| 12 | 1.0 | 6.6 | 0.1 | 36.6 | 0.7 | 27.8 | 27.3 | 5.5 |
| 16 | 1.2 | 6.2 | 0.1 | 37.9 | 0.5 | 24.2 | 30.1 | 6.1 |
| 20 | 1.2 | 6.0 | 0.1 | 36.6 | 0.4 | 22.4 | 33.5 | 6.1 |
| 24 | 1.1 | 5.8 | 0.1 | 35.4 | 0.4 | 21.9 | 35.3 | 6.1 |

Catalyst MgZSM-5(260), T = 470° C.

From both experiments it is observable that, for the MgZSM-5 catalyst having a Si/Al ration of 260, propylene yields increase with increasing WHSV=2 until WHSV=16, then slightly drops. C3⁼/C2⁼ increases with WHSV to almost a constant value (6.1). In addition, one also sees the effect of adding a Group II dopant such as Mg to the system, where the yield and C3⁼/C2⁼ both increase from those with the Mg free catalyst. Data such as the above helps guide further experimentation in accordance with the method of the invention to define a commercial scale system in which all of the series-connected reactors operate at or near optimal practical conditions.

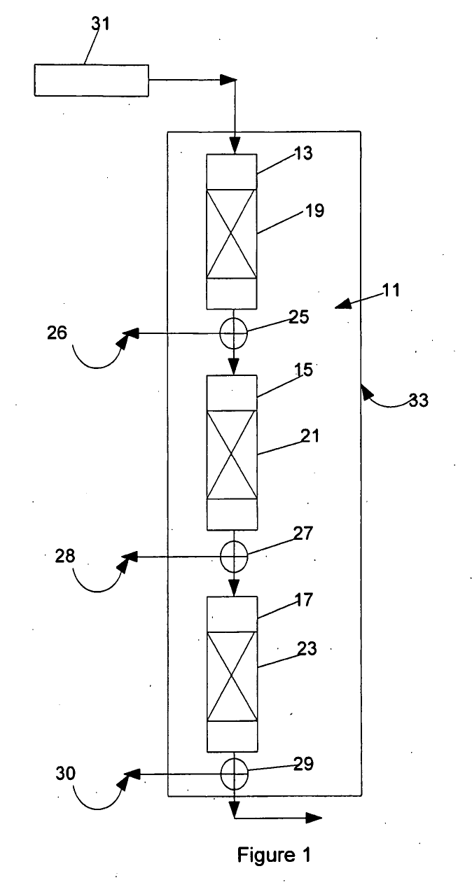

Referring to FIG. 1 of the drawings, the composite multistage laboratory scale plug-flow reactor 11 of a first embodiment of a reactor useful in performing the method of the invention is made up of three series-connected stages 13, 15 and 17 each of which contains a bed of catalyst particles 19, 21 and 23. A sampling valve 25 is connected between the output of the first reactor stage 13 and the input to the second reactor stage 15 and has an output 26 for sampling the effluent from the first reactor stage 13 for analysis. A sampling valve 27 is connected between the output of the second fixed bed reactor stage 15 and the input to the third fixed bed reactor stage 17 and has an output 28 for sampling the effluent from the second reactor stage 15 for analysis. A sampling valve 29 is connected to the output of the third fixed bed reactor stage 17 and has an output 30 for sampling the effluent of the third reactor stage 17 for analysis. The output of the third reactor stage 17 is connected through the valve 29 to, e.g., a product accumulator (not shown). The feed to the multistage fixed bed reactor 11, which normally is fresh reactant feed, is connected to the inlet of the first fixed bed reactor stage 13 from a source 31. A sampling valve may also be installed in the line between the feed source 31 and the inlet to the first fixed bed reactor stage 13 in order to permit analysis of the feed.

The multistage fixed bed reactor 11 is contained in a temperature control device 33 that, for the exothermic methanol and/or DME conversion reaction, can contain a material, such as circulating boiling water, for extracting heat from the reactor 11 in order to maintain the multistage reactor 11 at a substantially constant temperature. Other forms of temperature control device 33 can also be used for extracting heat from the reactor 11 to retain it at a substantially constant temperature. For instance, the temperature control device 33 can include a fluidized sand bath heater in which the multistage reactors are immersed.

Each of the catalyst beds 19, 21, and 23 in the reactor stages of the multistage reactor 11 replicates a longitudinal portion of the catalyst bed of a large plug-flow reactor or of one of the catalyst beds in a set of series-connected plug-flow reactors, and permits the measurement and analysis of the characteristics and performance of successive longitudinal portions of a large catalyst bed, or of one of the catalyst bed in a set of catalyst beds, thereby allowing determination of longitudinal gradients in reactor characteristics and performance that heretofore have been inaccessible. While reactor 11 has been shown as having three series-connected stages, it is equally possible to have a larger number of series-connected stages, e.g., four or six stages, in order to analyze the performance of the composite catalyst bed or set of beds at a greater number of points along its length.

For the study of the zeolite catalyzed conversion of methanol and/or DME to propylene and other unsaturates, the analysis of the feed and the effluent from the reactor stages can include, e.g., conventional GC/MS or UV or IR characterization of the reactant and product stream(s), and/or analysis of the catalyst system by XRD, diffuse reflectance IR or other spectroscopic techniques that are well known in the art. These studies would allow the performance attributes of the system to be quantified as a function of the longitudinal position in the catalyst bed or set of beds. Such knowledge allows the system to be optimized with direct knowledge of the catalytic reaction kinetics and performance attributes of each point and permits the design of catalyst systems in which, e.g., the catalyst particles may have different chemical or physical characteristics in different longitudinal portions of the catalyst bed or set of beds so as to operate at peak productivity or selectivity to propylene as a function of the local environment.

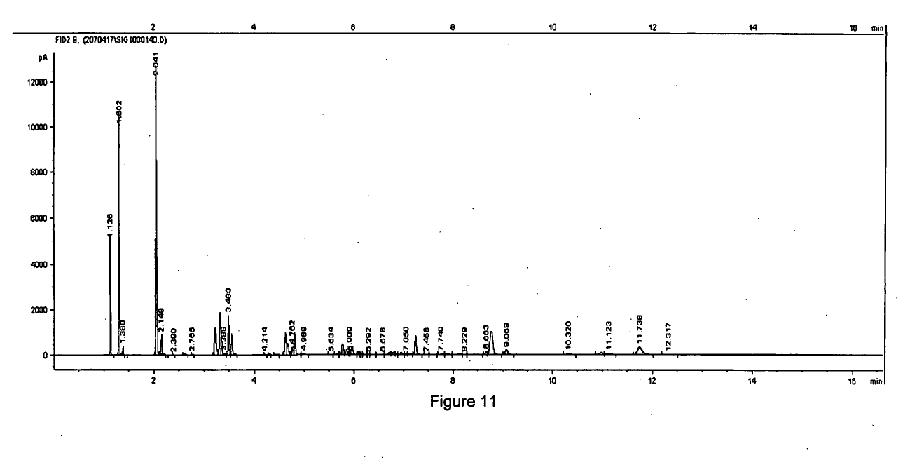

In one example of a full product analysis, an Agilent 6890 GC was used. An online analysis method was conducted using a PLOT-Q capillary column and a FID to perform a full analysis of all product components. FIG. 11 shows a typical chromatograph of the reactor down-stream components analyzed by Agilent 6890 GC. All components are well separated in the chromatograph, and allow quantitative determinations of their amounts.

The catalyst beds in the reactor stages 13, 15 and 17 may be a crushed or powdered catalyst or a commercial-size catalyst. Most measurements made in gathering data for the scale up of a catalytic reactor are preferably made with the reactor operating in a substantially isothermal regime. In order for the reactor stages 13, 15 and 17 to operate in a substantially isothermal regime, the cobalt-based catalysts in the beds 19, 21 and 23 are diluted with an inert particulate matter, typically in a ratio of up to about 8-10 to 1. For measurements being made with the reactor operating in a substantially adiabatic regime, the catalyst in the beds 19, 21 and 23 is less diluted, and depends on the heat of reaction of the process under study and reactor diameter. The ratio of catalyst particles to diluent particles in a catalyst bed depends upon a number of factors, including the amount of heat generated by the reaction and the activity of the catalyst particles in the bed. The appropriate ratio for a given reaction, catalyst, reactor diameter and catalyst particle size can easily be determined by one of ordinary skill in the art by a simple experiment.

A commercial-size catalyst in a fixed bed reactor typically has particle size of about 1 to 5 mm. the catalyst particles can be in any one or more a variety of shapes, e.g., round, tubular, trilobe, toroidal, etc. The crushed or powdered catalyst, which is typically formed by crushing a commercial-size catalyst, typically has a particle size of about 0.10-0.20 mm. the crushed or powdered catalyst particles are normally preferably as small as can be obtained while still retaining a performance qualities of the catalyst. The interior diameter of a reactor stage should be about 10 times the diameter of the smaller of the diluent or catalyst particles and the minimum would typically be in the range of about 10 to 50 mm (0.4 to 2 inches) for a bed containing commercial-size catalyst particles and diluent. Crushed or powdered catalyst particles are typically more active than the commercial-size catalyst particles because of lower mass transfer resistance. Therefore, in order for a reactor containing a bed of crushed or powdered catalyst to operate at the same temperature as a similar reactor containing commercial-size catalyst, the ratio of inert diluent particles to catalyst particles in the bed of crushed or powdered catalyst particles normally needs to be higher than that of the bed containing commercial-size catalyst particles in order that the heat release per unit volume of the to catalyst beds is the same.

The interior diameter of a reactor containing crushed catalyst, can, if desired, be smaller, in the range of about 5 to 12 mm, than that of a reactor containing the commercial size catalyst. For reasons of flexibility in the use of the multistage reactor 11 in different applications, however, it may be preferable that the crushed catalyst bed have the same interior diameter as that required for a bed containing commercial-size catalyst particles. Alternatively, the interior diameter of a reactor being used with a bed of crushed or powdered catalyst particles may be reduced by the use of a thermally conductive sleeve within the reactor.

The minimum height of a reactor stage is determined either by mixing or heat release considerations. For isothermal operation, if mixing is the limiting factor, the height should be sufficient to avoid bypassing. Typically, this would be at least about 50 times the average diameter of the particles, or about 50 to 250 mm (2 to 10 inches) for a reactor stage containing a bed of commercial-size catalyst particles. Because the feed is progressively converted as it traverses the stage of the multistage reactor 11, the concentration of fresh feed in the successive reactor stages decreases from one stage to the next. If it is desired to have constant conversion in each reactor stage, the lengths of the catalyst beds 19-23 can be progressively longer in each of the successive reactor stages 13-17. If the reactor 11 is to operate in the adiabatic regime, one would tend to use a lower ratio of inert diluent and a larger diameter reactor.

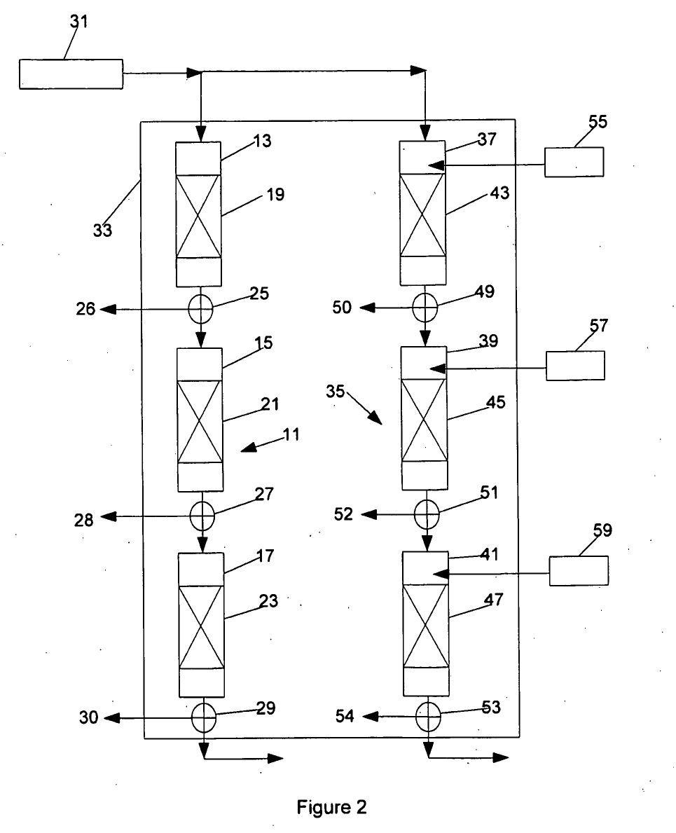

Referring to FIG. 2 of the drawings, there is illustrated a second embodiment of a reactor useful in performing the method of the invention in which elements that are the same as in the embodiment illustrated in FIG. 1 are numbered similarly. This second embodiment includes a composite multistage reactor 11 that is the same as the multistage reactor 11 of FIG. 1. A composite multistage probe reactor 35, in which each reactor stage can be the same as the corresponding reactor stage of multistage reactor 11, is operated in parallel with the multistage reactor 11. Both of the multistage reactor 11 and the probe reactor 35 are contained in a temperature control device 33 that can be the same as the types discussed above. If desired, the probe reactor 35 can be contained in a temperature control device separate from the temperature control device 33 in which the reactor 11 is contained, thereby permitting the operation of the probe reactor 35 at a temperature different from that of the multistage reactor 11.

The composite reactor 35 has three series-connected reactor stages 37, 39, and 41 that contain catalyst beds 43, 45 and 47, respectively. A sampling valve 49 is connected between the output of probe reactor stage 37 in the inlet of the probe reactor stage 39 and has an output 50 for sampling the effluent from reactor stage 37. A sampling valve 51 is connected between the output of reactor stage 39 and the input of reactor stage 41 and has an output 52 for sampling the effluent from the reactor stage 39. A sampling valve 53 is connected between the output of reactor stage 41 and, e.g., a product accumulator (not shown), and has an output 54 for sampling the effluent from reactor stage 41. The fresh reactant feed from source 31 is connected to the inlet of the first probe reactor stage 37. A control and sampling valve can be connected between the source 31 in the inlet to the first probe reactor stage 37 for selectively controlling the amount of feed to the probe reactor and to permit the sampling of the feed for analysis. Also connected to the inlet to the first probe reactor stage 37 is a source 55 of a material to be controllably added to the input of the first probe reactor stage 37 for ascertaining the effect of such addition on the characteristics and performance of the stages of the probe reactor 35. A source 57 is connected to the inlet of the second probe reactor stage 39 for selectively adding a material to the input of such a second probe reactor stage for ascertaining the effect of such addition on the characteristics and performance of the second and third probe reactor stages 39 and 41. A source 59 is connected to the input of the third probe reactor stage 41 for selectively adding a material to the input of such probe reactor stage for ascertaining the effect of such addition on the characteristics and performance of the third probe reactor stage 41. In this embodiment of the invention, the catalyst beds 43, 45 and 47 of the probe reactor 35 are preferably the same as the catalyst beds 19, 21 and 23 of the multistage reactor 11, respectively.

The use of the composite multistage probe reactor 35 allows one to measure the transient response of the system to permanent or temporary changes in the feed composition at any stage of the multistage reactor 11 by comparing the characteristics and performance of the relevant stages of the probe reactor 35 over time in response to the change in input with the characteristics and performance of the corresponding stages of the multistage reactor 11. Introduction of a change in gas or liquid input to the third reactor stage of the probe reactor 35 allows one to measure the impact of the changed component on the reaction rate and selectivity of the third reactor stage catalyst bed of the multistage reactor 11 with time. Introduction of the change to the second probe reactor stage allows one to measure the impact on the second and third stage catalyst beds of the multistage reactor 11. This is equivalent to measuring the response to a change in conditions of any small segment of the catalyst bed in a commercial-size fixed bed reactor. For example, raising the gas feed rate to any reactor stage of the probe reactor 35 by having one of the sources 55, 57 or 59 and additional fresh feed into the stage of the probe reactor 35 to which it is connected, would allow the investigation of the changes in incremental performance of that stage and following stages resulting from the change in input over time.

It is also possible to use the sources 55, 57 or 59 to vary the concentrations of the trace components present in the fresh feed in a selected probe reactor stage, for instance by adding fresh reactant feed having a higher or lower concentration of such trace components, in order to quantify the effect of such trace components on various parts of the composite catalyst bed under a full range of operating conditions. By doing this it would be possible to map the critical longitudinal portions of the composite catalyst bed in a commercial system in which the catalyst is most vulnerable to poisoning or other inhibitory reactions caused by poisons or other natural byproducts of the reaction being practiced. The probe reactor 35, and other versions of probe reactor as discussed below with relation to other Figures, can also be used to investigate the transient response of a reactor to temporary changes in the composition of the feed or prior stage effluent to various points in a composite catalyst bed by temporarily adding the materials of interest to a selected stage of the probe reactor 35 and monitoring the time dependent response of that stage and following stages of the probe reactor 35 to such added materials both during and after the time that such materials are added.

Referring to FIG. 3 of the drawings, there is illustrated another embodiment of a reactor useful in performing the method of the invention in which elements that are the same as in the embodiments of FIG. 1 are numbered similarly. In this embodiment, the probe reactor 101 can consist of a single fixed bed reactor stage whose inlet is selectively fluid dynamically linked to a selected stage of the composite multistage fixed bed reactor 11. Other configurations for the single stage probe reactor 101 are discussed below. The valve 103 is connected between the output of the first reactor stage 13 and the input of the second reactor stage 15 of the multistage reactor 11 and has outputs 105 and 107 for selectively sampling of the effluent of the reactor stage 13 and selectively connecting a portion of the effluent of the reactor stage 13 to the input of the probe reactor 101, respectively. The valve 109 is connected between the output of the reactor stage 15 and the input to the reactor stage 17 of the multistage reactor 11 and has outputs 111 and 113 for selectively sampling of the effluent of reactor stage 15 and selectively connecting a portion of the effluent of reactor stage 15 to the input of probe reactor 101, respectively. The valve 107 is connected between the output of reactor stage 15 and a product accumulator and has outputs 117 and 119 for selectively sampling of the effluent of reactor stage 15 and selectively connecting a portion of the effluent of reactor stage 15 to the input of probe reactor 101, respectively. The probe reactor 101 also receives inputs from the feed source 31 and from a source 121. The probe reactor 101 and the catalyst bed contained therein in this embodiment of the invention is preferably the same as the reactor stage and catalyst bed contained therein in the multistage reactor 11 following the one having a portion of its effluent connected to the input of the probe reactor 101. The single stage probe reactor may, for example, be used to perform the same investigations as were described above with relation to the multistage probe reactor embodiment of FIG. 2.

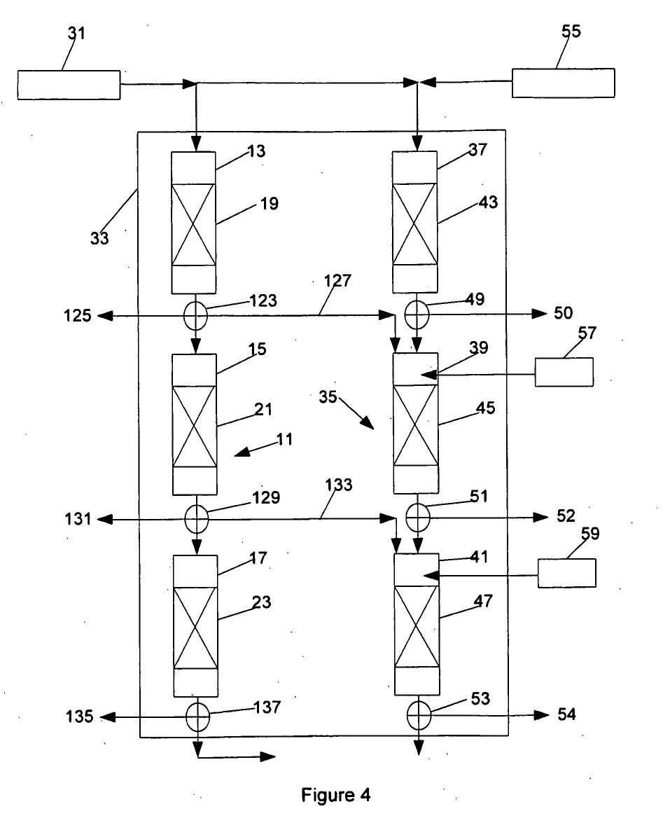

Referring to FIG. 4 of the drawings, there is illustrated another embodiment of a reactor useful in performing the method of the invention in which elements that are common to the embodiments of FIGS. 1 and 2 are numbered similarly. In this embodiment, the probe reactor 35 consists of a composite multistage series-connected laboratory scale plug-flow reactor in which the reactor stages may be the same as the multistage series-connected probe reactor 35 depicted in FIG. 2 of the drawings. In this embodiment, however, the stages of the probe reactor 35 are selectively fluid dynamically linked to selected stages of the composites multistage series-connected reactor 11 by selectively connecting a portion of the effluent of one or more stages of the composite multistage series-connected reactor 11 to one or more selected stages of the probe reactor 35. The valve 123 is connected between the output of the first reactor stage 13 and the input of the second reactor stage 15 of the multistage reactor 11 and has outputs 125 and 127 for selectively sampling the effluent of the first reactor stage 13 and connecting a selected portion of the effluent of reactor stage 13 to the inlet of probe reactor stage 39, respectively. The valve 129 is connected between the output of reactor stage 15 and the input to reactor stage 17 of the multistage reactor 11 and has outputs 131 and 133 for selectively sampling the effluent of reactor stage 15 and selectively connecting a portion of the effluent of reactor stage 15 to the input of probe reactor stage 41, respectively. The fresh reactant feed from the source 31 is connected to the input of the first probe reactor stage 37. Control and sampling valves (not shown) may be connected in the line between the fresh reactant feed and the probe reactor stage 37 to control the amount of fresh reactant feed supplied to the probe reactor 35 and to permit the analysis of its content. Also connected to the input to the first probe reactor stage 37 is a source 55 of a material to the selectively added to the input of the first probe reactor stage 37 for ascertaining the effect of such addition to the stages of the probe reactor 35. A source 57 is connected to the input of the second program per stage 39 for selectively adding a material to the input of such a second program per stage for ascertaining the effect of such addition on the second and third probe reactor stages 39 and 41. A source 59 is connected to the input of the third probe reactor stage 41 for selectively adding a material to the input of such probe reactor stage for ascertaining the effect of such addition on the third probe reactor stage 41. In this embodiment of the invention, the catalyst beds 43, 45 and 47 of the probe reactor 35 are preferably the same as the catalyst beds 19, 21 and 23 of the multistage reactor 11, respectively.

Referring again to FIG. 3 of the drawings, the probe reactor 101 can consist of a substantially fully back-mixed reactor instead of a single stage fixed bed reactor stage 101, such as discussed above. The distribution a catalyst, feed and products in the back-mixed probe reactor 101 a substantially uniform and so, if the probe reactor 101 receives only effluent from a stage of reactor 11, it corresponds to a single, narrow, horizontal slice at the inlet of the catalyst bed of the stage of multistage reactor 11 following the stage that has a portion of its effluent connected to the input of the probe reactor 101. By controlling the relative concentrations of fixed bed reactor stage effluent and fresh feed, it will is possible for the back-mixed probe reactor to simulate any selected horizontal slice of the fixed bed reactor stage whose effluent is connected to the back-mixed probe reactor. The back-mixed probe reactor 101 can, for instance, be a two-phase fluidized bed reactor, a three-phase slurry reactor, or a three phase ebulated bed reactor.

In the embodiments of FIGS. 2, 3 and 4 of the drawings, stages of the probe reactor 101 and 35 receive as inputs combinations of controlled amounts of one or more of the fresh reactant feed, effluent from a selected stage of the multistage reactor 11 and other feeds. Such other feeds may, for instance, consist of additional fresh reactant feed, additional product gases or liquids produced during the reaction taking place in the composite multistage reactor 11, or contaminants that may be present in the fresh feed used during operation of a commercial reactor.

The reactant and other material feeds, and reaction products and byproducts in reactor effluents supplied or generated in the embodiments of the invention described herein may be either gaseous, liquid or mixed phase (such as e.g., gas/liquid or two or more immiscible liquids). Feeds and effluents consisting of gases can be handled using well known conventional back pressure regulators and gas flow control systems with mass flow controllers. Controlled amounts of liquids can be pumped in high-pressure environments using known pumps such as a Ruska pump or a Syringe pump. If the effluent from a reactor stage or the feed contains multiple phases, particularly if such phases are immiscible, such as water and hydrocarbons or liquid and gas, it is important to avoid slug flow. In such case, sampling valves may consist of e.g., iso-kinetic sampling valves such as available from Prosery AS, or splitters such as described in U.S. Pat. No. 4,035,168. Alternatively, the stream may be sampled immediately after a static mixer such as available from Prosery AS, which homogenizes the multiphase stream. In combining immiscible feeds or feeds and effluent to a reactor stage, or in conducting the multiphase effluent from the outlet of one reactor stage to the inlet of the following reactor stage in a series-connected multistage reactor, it is typically the practice to manifold of the streams into a line having a high Reynolds number similar in concept to a fuel injection system in an automobile engine. Alternatively, static mixers such as available from Prosery AS or from Admix, Inc., Manchester, N.H., can also be used.

In the plug-flow zeolite catalyzed conversion of methanol and/or DME to propylene, deactivation can occur by a number of mechanisms which may vary as a function of the catalyst's longitudinal position in the reactor. The presence of unsaturated molecules, which are primary products in the DME conversion step, can lead to formation of carbon containing overlayers which retard overall catalytic activity; and the nature and reactivity of these carbon phases can vary as a function of the conditions under which they were formed. The nature and effect of various modes of deactivation on the performance of different segments of the fixed bed system is not well understood, and ways to mitigate deactivation by controlling a catalyst's chemical or physical properties or by adding other substances that will minimize their ability to form coke or to facilitate the removal of coke once formed, is of high importance. The present invention provides for a method for using a laboratory scale multistage plug-flow reactor to investigate the overall mechanism of deactivation as a function of longitudinal position within a full scale reactor, and provides a means for developing improved regeneration methods without the need for full scale operations.

The method of the current invention using composite multistage series-connected laboratory scale plug-flow reactors, enables the investigation of catalyst deactivation under normal operating methanol and/or DME to propylene conditions, and under conditions that simulate specific deactivation agents and pathways by addition of external agents such as olefins, dienes, acetylenes, aromatics and other unsaturates to the feed gas for the methanol and/or DME to propylene process. The overall degree or percent of deactivation can be varied by changing the operating conditions such as temperature, flow rate, feed gas composition, pressure for the methanol and/or DME to propylene process, or the time under which deactivation occurs. By taking this approach it is possible to study the extent and reversibility of deactivation as a function of the degree of deactivation that has occurred or to study the nature of the deactivation process as a function of the position of the catalyst in the composite catalyst bed of the reactor system.

In accordance with the invention, the catalyst is subjected to conditions under which deactivation occurs, the extent of deactivation is quantified, the catalyst is then subjected to some form of regeneration process, and the extent to which activity or other performance properties were reacquired is determined by some form of methanol and/or DME to propylene activity/performance test. The catalyst can also be subjected to some form or surface or bulk analytical characterization method to determine the nature of chemical or physical property changes that occurred during the deactivation process and/or during the regeneration process. The procedure thus allows one to determine the nature of deactivation as function of catalyst position within the reactor and the efficacy of various regeneration procedures for that mode of deactivation. This information provides the basis for developing an integrated method for regenerating a methanol and/or DME to propylene catalyst system in a full scale system without the need for operating in a full scale PDU or larger reactor system.

A major area of concern in understanding and controlling the characteristics and performance of a fixed bed reactor is the adsorption or reaction of a feed component, product or byproduct with the catalyst surface. For instance, in the zeolite catalyzed conversion of methanol and/or DME to propylene or higher olefins, materials such as olefins, aromatics, ammonia, carbon monoxide and hydrogen sulfide can tie up active catalyst sites, reduce reaction rate and adversely impact product selectivity. The reactions caused by these materials can take time to equilibrate and can also take time to be released after removal of the material from the feed stream to the reactor.

Ammonia is known to react with acid sites on zeolites, causing activity to vary. Upon removal of the ammonia (or other heteroatom containing molecules) from the feed, hydrogen can be used to remove the ammonia from the catalyst surface. In investigating the effects of ammonia on different portions of the composite catalyst bed, ammonia can be added to the inlet of any of the stages of probe reactor, thereby replicating the effect of the presence of ammonia in the feed to a selected longitudinal slice of the composite catalyst bed. By controlling the conversion level in a given catalyst slice, e.g., by adjusting temperature and/or flow rate and/or reactant partial pressures in a probe reactor stage, it is possible to define the effect of the ammonia under various operating conditions. By varying the hydrogen concentration in the feed to one or more probe reactor stages, it is possible, for example, to investigate the effect of increased hydrogen on the ammonia-contaminated catalyst in different portions of the composite catalyst bed, e.g., the bed with the greatest activity decline.

Aromatics can be tightly held on a zeolite catalyst for conversion of methanol and/or DME to propylene, which can reduce available surface for alcohol or ether activation, thereby making chemisorption of the feed the rate limiting step. By varying the concentrations of aromatics (or other unsaturates) in the feed to selected stages of the probe reactor 35 or 101 and comparing performance of the relevant probe reactor stages with the corresponding stages of the multistage reactor 11, it is possible to determine the impact of these molecules on reaction rate and selectivity. The use of a multi-stage probe reactor allows for testing of the impact at various conversion levels by e.g., by adjusting temperature and/or flow rate and/or reactant partial pressures.

The addition of steam to a fixed bed reactor in zeolite catalyzed conversion of methanol or DME to propylene or higher olefins is believed to have a positive impact on reaction rate under some conditions, while having a negative effect under other conditions. Adding controlled amounts of steam or other oxygen containing molecules to selected stages of the probe reactor 35 or 101 would permit the study of the impact of the added water on reaction rate and selectivity in selected longitudinal slices of the composite catalyst bed by comparing the characteristics and performance of the relevant stages of the probe reactor with the corresponding stages of the multistage reactor 11.

Surface or carbidic carbon present on a zeolite catalyst for conversion of methanol or DME to propylene or higher olefins will usually affect activity in a negative way. Heavier unsaturates present on or in a catalyst particle has a similar impact on their performance; and may represent a key pathway for catalyst deactivation. In general, carbon and heavy hydrocarbon deposits on a catalyst inhibit the diffusion of reactants to the catalyst surface and the removal of reaction products from the catalyst surface. This tends to lead to activity reduction via unwanted side reactions with deposits on the catalyst surface or with the diffusion limited reactants or both. In the case of beds containing commercial-size catalyst particles where the diffusion path is the longest, this sort of diffusion limitation can limit overall catalyst life and require costly steps to maintain system performance. Adding different molecular weight fractions of aromatics or other unsaturates to the zeolite catalyst bed or at a selected stage of the probe reactor 35 or 101 allows the determination of what portion of the composite catalyst bed is impacted the most. The effects of various regeneration techniques such as by the addition of hydrogen, steam, oxygen or a light solvent can also be determined by controlling the feeds to the relevant stages of the probe reactor 35 or 101, thereby to define the preferred regeneration technique.

Referring now to FIG. 5 of the drawings, the series-connected reactor stages of the composite multistage fixed bed reactor can be arranged in parallel with one another in a temperature control device for a more compact and convenient configuration. In this arrangement the composite multistage laboratory scale reactor 501 is made up of three series-connected reactor stages 503, 505 and 507 which are disposed in temperature control device constituted by a heated or cooled fluidized sand bath 509. The reactant feed gas is preferably connected from a source file a through a preheat coil 513, which is also disposed in the fluidized sand bath 509, to the inlet of the first reactor stage 503. Any liquid is fed from the feed pump 515 through the preheat coil 513 to the inlet of reactor 503. Sampling valves may be connected in the both the gas and liquid feed lines for sampling the gas and liquid feeds. The preheat coil 513 is used to heat the gas and liquid feeds to the appropriate temperature for being supplied to the multistage reactor 501. The outlet of reactor 501 is connected to the inlet of reactor 505 through a sampling valve 517. The outlet of reactor stage 505 is connected to the inlet of reactor stage 507 through the sampling valve 519, and the outlet of reactor stage 507 is connected through a sampling valve 521 to the separator 523. Each of the sampling valves 517, 519 and 521 have an outlet selectively connected to a probe reactor 523 for supplying effluent to the probe actor 523. Each of the sampling valves 517, 519 and 521 also has an outlet to permit sampling of the effluent from the respective reactor stage to whose output of the sampling valve is connected.

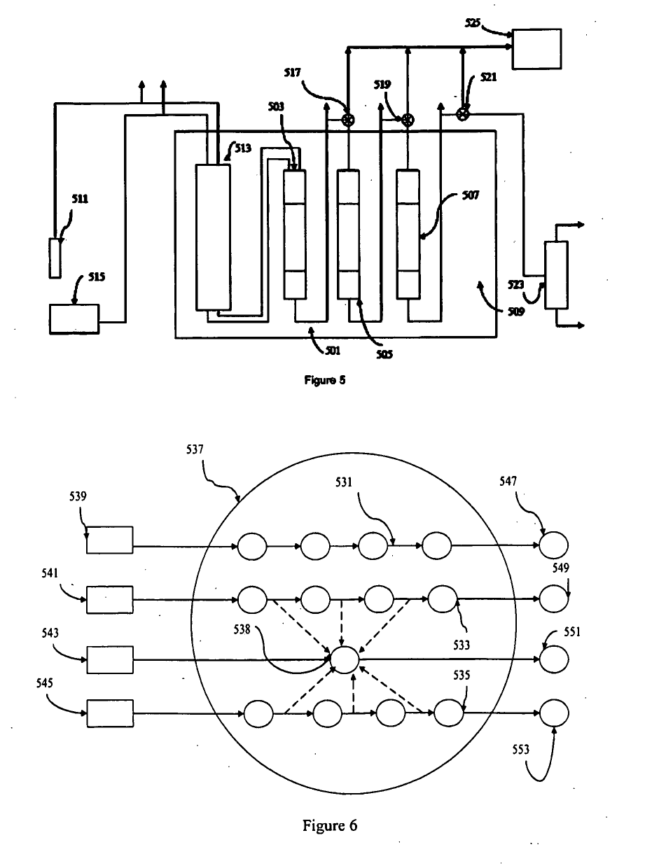

Referring now to FIG. 6 of the drawings, there is illustrated schematically, an arrangement of three composite multistage series-connected laboratory scale fixed bed reactors 531, 533 and 535 and arranged in a fluidized sand bath 537. The stages of each of the multistage reactors are arranged in parallel with one another in the same manner as the stages of the reactor 501 in FIG. 5, and each of the multistage reactors 531, 533 and 534 is preferably preceded by a preheat coil that can be the same as the preheat coil 513 illustrated in FIG. 5 of the drawings. A single-stage probe reactor 538, which can be any of the types described above with relation to probe reactor 101 of FIG. 3 of the drawings, is arranged between the series-connected reactors 533 and 535 in the fluidized sand bath 537 and selectively receives inputs of either the reactant feed or the effluent of any of the reactor stages of the series connected reactors 533 and 535 by means of sampling valves (not shown) that may be the same as the sampling valves 517, 519 and 521 illustrated in FIG. 5 of the drawings. Each of the reactors 531, 533, 538 and 535 receives reactant feed from sources 539, 541, 543, and 545, respectively, that can be all the same feed source. The outlets of the last stages of each of the reactors 531, 533, 538, and 535 are preferably connected to the separators or product accumulators 547, 549, 551 and 553, respectively, which all may be constituted by a single separator or product accumulator.

The arrangements of FIGS. 5 and 6 have the advantage that the fluidized sand bath need not be so deep as it would be if the reactors were arranged vertically, and in that the sampling valves 517, 519 and 521 can be situated above the fluidized sand bath and so are accessible for maintenance or adjustment during operation of the multistage reactors. If the effluents from the stages of the multistage reactors contain multiple phases, the transfer lines connecting the outlet of one reactor stage to the inlet of the following reactor stage need to be configured in such a way as to avoid a slug flow in the lines. As described above, this can be accomplished using lines having high Reynolds numbers or with the use of static mixers. The sampling valves 517, 519 and 521 can be iso-kinetic sampling valves, although other arrangements such as described above can also be used. Additionally, the conduits connecting the outlet of one reactor stage to the inlet of the following series-connected reactor stage are designed for non-slugging flow, for instance by using static mixers.

Having a plurality of composite multistage series-connected reactors disposed in a common temperature environment, such as constituted by the fluidized sand bath 537, or as described above with relation to FIGS. 2 through 4 of the drawings, permits the simultaneous investigation of various characteristics of a catalytic process for substantially accelerating the scaling up of the reaction to commercial application. For instance, using the configuration of FIGS. 5 and 6 as an example, if the multistage reactor 535 contains crushed catalyst particles diluted with an inert diluent for isothermal operation, and the reactor 533 contains commercial scale catalyst particles also diluted with an inert diluent for isothermal operation, and the reactor 531 contains commercial scale catalyst particles in a concentration suitable for adiabatic operation, the kinetic, mass transfer and heat transfer characteristics of the catalytic process can be investigated simultaneously in the isothermal reactors, and the resulting reactor model derived from the data obtained from the isothermal reactors can be confirmed by the data obtained from the adiabatic reactor.

Other experiments to be performed that aid in the scaling up of a catalytic process include, for example, investigating the characteristics of a plurality of different catalysts simultaneously. Alternatively, a crushed catalyst in the catalyst beds of one multistage series-connected reactor could be compared with a plurality of different shapes or sizes of commercial-size versions of the catalyst in the catalyst beds of other multistage series-connected reactors, all disposed in a common constant temperature department. In an alternative arrangement, it is also possible to have different catalysts in different reactor stages of the multistage series-connected reactor 11 for testing the catalysts in series. Using such an arrangement, one can design a layered composite catalyst bed in which the intrinsic behavior of each catalyst layer is matched to the local kinetic and mass transfer environment, so that the overall response of the system is varied longitudinally so as to obtain behavior characteristics in each longitudinal portion of the composite reactor that are optimum for process performance. If a plurality of multistage series-connected fixed bed reactors is disposed in separate, independently controllable temperature control devices, a plurality of different heat removal levels can be investigated in parallel.

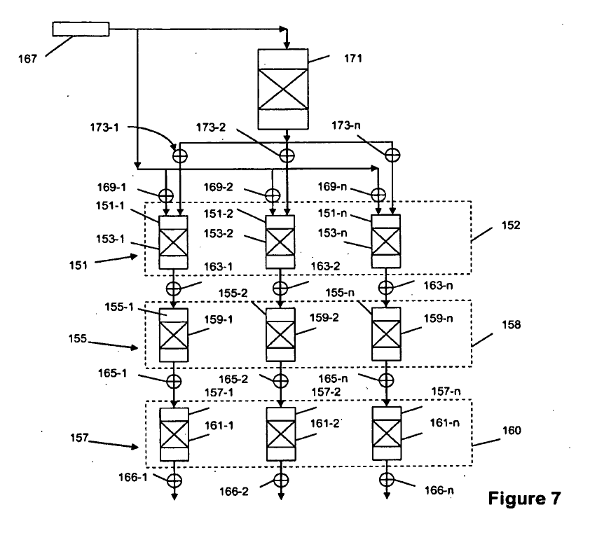

Referring now to FIG. 7 of the drawings, the module 151 contains a plurality of parallel laboratory scale fixed bed reactor stages 151-1 through 151-n. The module 151 includes a temperature control device 152 surrounding the module 151 for controlling the temperature of the ambient experienced by the reactor stages 151-1 through 151-n. In the case of an exothermic reaction, such as methanol and/or DME conversion to propylene, the temperature control device may consist of an enclosure containing circulating boiling water, or fluidized sand bath heater in which the multistage reactors are immersed, for extracting heat from the reactor stages 151-1 through 151-n.

Each of the reactor stages 151-1 through 151-n contain a catalyst bed 153-1 through 153-n. The modules 155 and 157 can be identical to the module 151, and contain a plurality of parallel fixed bed reactor stages 155-1 through 155-n and 157-1 through 157-n, respectively. Each of the parallel reactor stages in the modules 155 and 157 contain catalyst beds 159-1 through 159-n and 161-1 through 161-n, respectively. In the illustrated embodiment, the outlet of each of the reactor stages in module 151 is connected to the inlet of the corresponding reactor stage in module 155, and the outlet of each of the reactor stages in module 155 is connected to the inlet of the corresponding reactor stage in module 157. Thus, the series-connected reactors stages 151-1, 155-1 and 157-1 form a composite multistage series-connected fixed bed reactor. Similarly, the other sets of series connected reactor stages in the modules 151, 155 and 157 also form composite multistage series-connected fixed bed reactors. The modules 151, 155 and 157 may contain any desired number of parallel reactor stages depending upon the application. For instance, each module might contain four or eight or even 16 parallel reactor stages. Is also possible to have additional modules of parallel reactors stages, with each of said parallel reactors stages being connected in series with the corresponding reactor stages of the preceding and succeeding modules. For instance, there might be four or six modules in a given application.

The modules 155 and 157 are surrounded by temperature control devices 158 and 160, respectively, that may be the same as, or common with, the temperature control device 152 that surrounds the module 151. Sampling valves 163-1 through 163-n are connected between the outlet of each reactor stage in the module 151 and the inlet of the corresponding reactor stage in module 155. Sampling valves at 165-1 through 165-n are connected between the outlets of each of the reactor stages in module 155 in the inlet of the corresponding reactor stage in module 157. Fresh reactant feed is fed from a source 167 through control valves 169-1 through 169-n to the inlets of each of the reactor stages 151-1 through 151-n of module 151 for supplying controlled amounts of reactant feed to the inlets of the respective reactor stages. The fixed bed reactor 171 also receives fresh reactant feed gas from the source 167 at its inlet, and has its outlet connected to the inlets of the reactor stages 151-1 through 151-n through control valves 173-1 through 173-n, respectively, for supplying controlled amounts of effluent from the reactor 171 to the reactors 151-1 through 151-n.

In a commercial-size fixed bed reactor, the proportion of fresh feed and reaction products and byproducts varies continuously along the length of the catalyst bed. At the inlet there is 100% fresh reactant feed and zero reaction products and byproducts. As the fresh feed is consumed in the catalyst bed of the reactor, the proportion of fresh feed decreases and the proportion of reactant products and byproducts increases longitudinally along the catalyst bed. The multiple parallel-serial reactor arrangement of FIG. 7 can be used to perform a number of different kinds of experiments. For instance, all of the reactor stages can contain the same catalyst and the composition of the feed can be varied from stage to stage. Alternatively, the composition size or configuration of the catalyst particles can be varied from reactor stage to reactor stage in each of the reactor stages can receive the same feed.

In accordance with the method of the invention, the rate of disappearance of methanol or DME reactants and appearance of products and byproducts of the reactions occurring are measured as they occur at successive points along the composite cobalt-based catalyst bed. In particular, at each reactor stage the relative amounts of olefins, aromatics, CO, CO₂H₂O and hydrocarbons are determined, e.g., by GC Mass Spectroscopy, or Quadripole Mass Spectroscopy. Tracer molecules, such as alkyl substituted olefins, aldehydes or ketones, or higher alcohols can be added to the reaction stream at selected points and in selected amounts for investigating system properties such as the kinetics of discrete reaction steps e.g., aromatization, and the relative structural sensitivity to changes in molecular shape or unsaturated content of the chemisorption sites on the catalyst particles, along the composite bed.

Using the above longitudinal data, the kinetics and mass transfer characteristics of the system can be investigated as they vary along the composite catalyst bed. The effects of physical variations in catalyst particles, such as particle size and shape and pore diameter and tortuosity can also be investigated longitudinally along the composite bed by repeating the measurements of the relative amounts of the zeolite catalyzed reaction products and byproducts present in the effluents of each of the reactor stages of the composite multistage series-connected fixed bed reactor with the catalyst beds containing catalyst particles having the relevant physical characteristics. For determining mass transfer characteristics, the catalyst beds in the reactor stages of the composite reactor during one of these sets of measurements preferably contains crushed or powdered catalyst particles.

In accordance with the method of the invention, the analysis of the system can also include an investigation of the structure—function effects of the zeolite catalyzed reaction e.g., redox states, crystalline phases of the zeolite or internal or external acid sites and corresponding oxides, carbides and protonated phases. Such investigation can also include the investigation of the service and bulk properties of the zeolite catalytic sites and their effects on the activity of the catalyst. For instance, the metallic cobalt at the catalytic sites on the catalyst particles can form inter-metallic oxides with the catalyst support particles that are not catalytically active. Additionally the individual acid catalytic sites on the catalyst particles can be affected thereby reducing the effective surface acidity, which results in a corresponding reduction in the catalyst activity. This investigation can be accomplished by investigating the catalyst particle characteristics in situ e.g., by XRay or Mossbauer Spectroscopy, or by measuring characteristics of the catalyst particles removed from a fixed bed reactor stage, such as a probe reactor. The measurements can be performed using various techniques, such as temperature programmed reduction or temperature programmed oxidation techniques, acid-base titration, ammonia chemisorption and surface spectroscopy techniques such as XRay absorption, surface enhanced Raman spectroscopy, or laser photoionization.

Kinetics

Heretofore, it has been the practice to measure the kinetics of a fixed bed catalytic system only by measurements taken at the inlet and the outlet of the catalyst bed, so that the measurements are averaged over the length of a catalyst bed. In analyzing the kinetic performance of such a reactor, it was necessary to make assumptions concerning the kinetic order of the reaction. Typically, it was assumed that the order of the reaction remained constant along the length of the catalyst bed in the reactor. Applicants have found that this assumption was in many cases incorrect. With the use of the multistage series-connected fixed bed reactor of the present invention as described above with relation to any of the FIGS. 1 through 7, it is possible to investigate longitudinal variations in the kinetics of a plug-flow catalytic system along the length of the catalyst bed of the reactor.

Using the multiple parallel-serial reactor arrangement illustrated in FIG. 7 of the drawings as an example, a multistage series-connected laboratory scale reactor can be used in accordance with the method of the invention to develop scale-up data for investigating the integral, differential and intrinsic kinetics of a plug-flow catalytic reactor system as a function of the longitudinal position along the catalyst bed of the reactor. For example, to determine the integral kinetics of a fixed bed reactor system, the catalyst beds in the reactor stages of modules 151, 155 and 157 and the reactor 171 can contain the catalyst intended for use with the system. The parallel reactor stages 151-1 through 151-n in the module 151 receive varying proportions of fresh feed from the source 167 and effluent from the reactor 171. For instance, the valves 169-1 through 169-n and valves at 173-1 through 173-n can be set such that reactor stage 151-1 receives 100% fresh feed and no effluent, and the reactor stages 151-2 through 151-n receive successively decreasing proportions of fresh feed and increasing proportions of effluent. In this arrangement, the successive reactor stages 151-1 through 151-n are equivalent to successive, longitudinally-spaced slices of the catalyst bed of a fixed bed reactor, with reactor stage 151-1 being equivalent to the slice at the inlet of the catalyst bed and reactor stages 151-2 through 151-n operating at conditions equivalent to slices of the catalyst bed positioned at successive longitudinal positions along the composite bed. The reactor stages in modules 155 and 157 can be used to provide data for slices of the catalyst bed being scaled-up that are intermediate the slices of the successive reactor stages in module 151. For example, if reactor 171 is operated at 90% conversion, its effluent will contain 10% of the amount of fresh feed at its inlet with the remainder of the effluent being reaction products and byproducts. If reactor stage 151-2 receives 88% fresh feed and 12% effluent from the reactor 171, the composition of the feed at the inlet to reactor stage 151-2 will be 89.2% fresh feed with the remainder being reaction products and byproducts. If the reactor stages 151-1, 155-1 and 157-1 are each run at 3% conversion, their effluents will contain 97% fresh feed, 94.1% fresh feed and 92.3% fresh feed, respectively, with the remainder being reaction products and byproducts. Thus, the compositions and proportions of fresh feed and reaction products and byproducts in the reactor stages in modules 151 155 and 157 are equivalent to those at successive longitudinal slices in the catalyst bed of a fixed bed reactor.

In order to determine the integral kinetics of the catalytic system formed by a composite multistage series-connected fixed bed reactor as a function of longitudinal positions in the catalyst bed, it is necessary to analyze the inlet feed stream and composition and outlet feed stream and composition, normalized, for instance to STP per standard liter of feed, at each of the successive longitudinal slices of the catalyst bed. For instance in a zeolite catalyzed reaction, one would measure how many moles of DME or methanol were consumed and how much product and byproduct were produced in each reactor stage. The conversion, or an equivalent quantity, such as the remaining concentration of fresh feed, is then plotted versus the residence time, which corresponds to successive longitudinal positions along the catalyst bed as the reactant feed traverses the catalyst bed. The slope at each point along the resulting curve is equal to the Reaction Rate for the system. The reaction rate is then plotted on a log-log plot versus the concentration of the fresh feed along the reactor catalyst bed. If the resulting curve is a straight line, the integral kinetics of the system is a constant along the length of the catalyst bed. If the line is horizontal, the system has first-order kinetics. If the line has a positive slope, the system has positive order kinetics greater than one. If the line has a negative slope, the system has negative order kinetics.

If the resulting curve on the log-log plot is not a straight line, then the integral kinetics of the system varies along the length of the reactor catalyst bed. In this case, it is necessary to do a regression analysis to fit the curve to an equation relating the reaction rate to the concentration of feed. Differentiating that equation, either graphically or mathematically, gives the Rate Model Correlation as a function of longitudinal position along the catalyst bed. A representative graphic technique is discussed in Graphical Methods for Data Analysis, John M. Chambers, Chapman and Hall, May 1983, ISBN: 0412052717.

In order to determine the effects of temperature and pressure on the integral kinetics of the system, the above-described experiment can be run at different temperatures and at different pressures. The experiment can also be run using different size catalysts. For example, the experiment can be run using the intended commercial size and shape catalyst and also with a diluted crushed or powdered catalyst.

The intrinsic and differential kinetics, free of mass transfer and heat transfer effects, of the composite multistage series-connected fixed bed catalytic system of the invention can also be investigated for purposes of scale-up to a commercial system using the systems depicted in FIGS. 1-7 of the drawings. Using the system depicted in FIG. 7 as an example, the catalyst beds of the reactor stages include very finely crushed or powdered catalyst particles in order to avoid mass transfer effects, and the catalyst is highly diluted to avoid heat transfer effects. Additionally, the diameter of the reactor should preferably be small, typically about 5 to 12 millimeters to further avoid heat transfer effects. This can be accomplished by using a smaller diameter reactor or by using a heat conductive sleeve in each reactor stage to reduce its diameter. The depth of the catalyst bed in each of the reactor stages is typically between about 5 and 10 centimeters. The same series of measurements and calculations are performed as described above for determining the integral kinetics of the system. In determining the differential kinetics of the system the amount of conversion in each reactor stage should be very small, e.g. less than 20 percent, preferably about 2 to 5 percent in the case of a methanol and/or DME conversion reaction. The measurements can be performed at different temperatures and pressures in order to investigate the effects of temperature and pressure on the intrinsic and differential kinetics of the system.

While these kinetics measurements have been described with relation to FIG. 7, it would also be possible to use the other disclosed reactor systems such as that described with relation to FIG. 1 or 5 of the drawings, using enough series-connected reactor stages to give the necessary of longitudinal information along the composite catalyst bed. A significant advantage of the system of FIG. 7 of the drawings is that the use of the reactor 171 to supply the effluent to all of the reactor stages in module 151 means that each of the reactor stages in the module 151 receives exactly the same reaction products and byproducts and trace elements, thereby replicating actual reactor conditions more exactly and eliminating errors resulting from variations in the composition of the feed to the reactor stages. Additionally, the composition of the inputs and outputs from all of the reactor stages can be sampled substantially simultaneously to give a snapshot of the reactor's performance at a given moment. The sampling of the composition of the inputs and outputs from the reactor stages can also be repeated periodically while the reactor system continues to operate thereby investigating the performance of the reactor system as a function of time on stream to see what aspects of the reactor performance change and in what longitudinal zones of the overall catalyst bed the changes occur. This data is useful in investigating the catalyst stability, among other things.

Mass Transfer

Methods of investigating the mass transfer characteristics of a catalytic process in a plug-flow reactor typically involve a comparing the conversion versus residence time characteristics at a given set of operating conditions of a finely crushed catalyst with that of a commercial-size catalyst. The crushed catalyst is screened to a narrow particle size range, preferably one that is close to the minimum obtainable catalyst particle size that still retains its catalytic properties. This minimum catalyst particle size depends on the characteristics of the specific catalyst being used, and can be determined by simple experimentation. In the more simple method for determining the mass transfer characteristics, the finely crushed and screened catalyst is assumed not to have any mass transfer limitations, so that any difference in the conversion versus residence time characteristics between the crushed catalyst and the commercial-size catalyst is assumed to be the result of mass transfer limitations. For a given feed, the effluent of the two reactors is sampled to determine the amount of conversion. Alternatively, the input flow rates of the two reactors can be adjusted (i.e., the input flow rate to the crushed catalyst in reactor is increased, or the input flow rate to the commercial-size catalyst reactor is decreased) so that each of the reactors has the same percentage conversion, and that difference in residence times is attributed to mass transfer limitations in the commercial-size catalyst.

In a more rigorous and technically exact method of determining the mass transfer characteristics of a commercial-size catalyst, the finely crushed catalyst is not assumed to have zero mass transfer limitations, and the Thiele Modulus of the commercial catalyst is determined from the ratio of the observed reaction rates of the crushed and commercial-size catalysts and the ratio of their particle sizes. The Effectiveness Factor for the commercial-size catalyst can then be determined from a plot of the effectiveness factor versus the Thiele Modulus. This method is described in Hougen and Watson, Chemical Process Principles, Part III, Kinetics and Catalysts, pp. 998-1000, Wiley, March 1966, which is incorporated herein by reference.

A problem with both of these methods is that they does not give any information concerning longitudinal variations in mass transfer performance along the reactor catalyst bed and basically assumes that the mass transfer characteristics are uniform from input to output. This assumption is incorrect for many catalytic systems, and the inability to investigate the longitudinal variations in mass transfer characteristics in a fixed catalyst bed has meant that information which would allow the optimization of the catalyst bed along its length has not been available.

In accordance with the method of the present invention, the catalyst beds of the fixed bed reactors are segmented longitudinally into at least three series-connected stages and the effluent of each of the stages is sampled to determine the amount of conversion occurring in each longitudinal segment of the catalyst bed. Referring again to FIG. 1 of the drawings, in accordance with the present invention, each of the reactors 11 and 35 includes three or more reactor stages with sampling valves between the output of each stage and the input of the succeeding stage for measuring the content of the effluent of each stage. The temperature control device 33 maintains both of the reactors 11 and 35 in a common thermal environment. The reactors 11 and 35 both receive the identical reactant input feed from the source 31. In performing a basic mass transfer investigation, the sources 55, 57 and 59 are preferably not used. The catalyst beds 19, 21 and 23 in reactor stages 13, 15 and 17 of reactor 11 contain a finely crushed and screened or powdered catalyst mixed with enough inert diluent particles so that the operation of the reactor 11 is essentially isothermal. Typically, in an exothermal reaction such as DME conversion, the ratio of diluent particles to crushed catalyst particles is up to about 10 to 1.

The catalyst beds 43, 45 and 47 in reactor stages 37, 39 and 41 of reactor 35 are composed of commercial-size catalyst particles that also may be mixed with a lesser percentage of inert diluent particles so that the operation of reactor 35 is also essentially isothermal. Typically, in an exothermic reaction such as methanol or DME conversion, the ratio of inert diluent particles to catalyst particles is about 1 to 1 up to about 10 to 1, and can be determined by simple experimentation.

To investigate the longitudinally-dependent mass transfer characteristics of the commercial-size catalyst in accordance with the method of the invention, each of the reactors 11 and 35 receive the identical reactant feed from the source 31 and the pressure and the feed rate for each of the two reactors is held constant. The conversion versus residence time relationship is obtained for each stage of the reactors 11 and 35 from the difference in the amount of reactant feed at the inlet and outlet of each reactor stage and the flow rate, for a given set of operating conditions.

In the simplified method of determining mass transfer limitations, the Effectiveness Factor for the commercial-size catalyst is obtained for the commercial-size catalyst at each stage of the reactor 35 by taking the ratio of the Observed Reaction Rates of the commercial-size catalyst and the crushed catalyst for each reactor stage. The Observed Reaction Rate is obtained for each reactor 11 and 35 by plotting the cumulative conversion of reactant and corresponding cumulative appearance of the product and byproducts (if any) versus residence times at the outputs of the reactor stages of each reactor and fitting curves to the data using well-known techniques. See, e.g., Graphical Methods for Data Analysis, John M. Chambers, Chapman and Hall, May 1983, ISBN: 0412052717. See also, A Mechanistic Study of Fischer-Tropsch synthesis using transient isotopic tracing. Part-1: Model identification and discrimination, van Dijk et al., Sections 3, 5 and 5.2. & FIG. 13. The slope of the resulting curve for the product at any residence time or conversion level for one of the reactors 11 or 35 is the Observed Reaction Rate, Kₒ (conversion per unit of residence time) for such reactor for such product. If mass transfer were not limiting, the Kₒ would be independent of particle diameter. A comparison of the plots of Kₒ versus conversion for the two reactors defines the longitudinal areas of the composite catalyst bed of the reactor 35 containing the commercial-size catalyst in which mass transfer through the catalyst pores is limiting. The Effectiveness Factor for a catalyst in a reactor is equal to the Kₒ divided by the Intrinsic Reaction Rate, K_{b} for such catalyst in the reactor. In the simplified method, the crushed catalyst is assumed not to have any mass transfer limitations, so that its Kₒ is equal to the Kᵢ for the catalyst. Therefore, the Effectiveness Factor for the commercial-size catalyst at any point along the composite catalyst bed of reactor 35 is equal to the ratio of the Kₒ of the commercial-size catalyst to that of the crushed catalyst at such point along the catalyst beds.

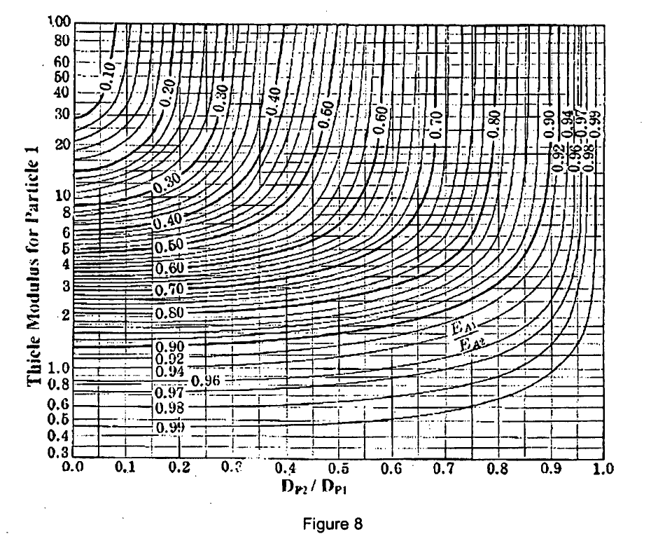

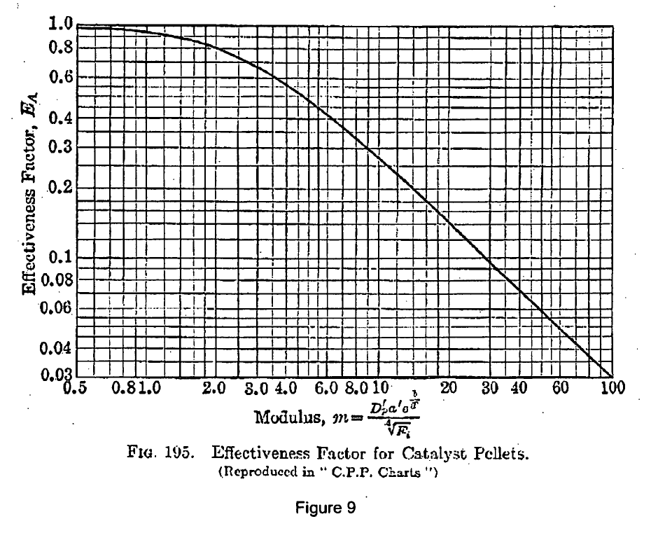

If the Hougen and Watson method is used, the Kₒ of the crushed catalyst is not assumed to be equal to the Kᵢ. According to this method, it is possible, using the graph of FIG. 8 of the drawings, to determine the Thiele Modulus for the commercial-size catalyst at any point along the catalyst bed from the ratio of Kₒ's at such point and the ratio of the particle diameters of the commercial-size and crushed catalysts. For instance, if the ratio of the particle diameter of the crushed catalyst to that of the commercial-size catalyst is 0.2, and the ratio of Kₒ of the commercial-size catalyst to that of the crushed catalyst is 0.34 at a given point along the catalyst beds, the Thiele Modulus at that point is about 9. Using the graph of FIG. 9, the Effectiveness Factor for the commercial-size catalyst at that point along the composite catalyst bed of reactor 35 is about 0.27. The determination of the longitudinally dependent Effectiveness Factor for the catalyst bed containing the commercial-size particles can be performed repeatedly during running of the reactors 11 and 35 to determine the effect of time on stream on the mass transfer characteristics of the fixed bed catalyst system. The measurements can also be repeated at different operating conditions of temperature and pressure in order to investigate the longitudinally dependent effects of changes in these parameters on the mass transfer characteristics of the composite catalyst bed of the fixed bed reactor 35.

Because the Effectiveness Factor is the ratio of Kₒ to the Kᵢ, it is possible to calculate the Kᵢ for a catalyst from the Effectiveness Factor and the Kₒ for a given longitudinal point along the catalyst bed. Since Kᵢ is the same for the crushed and commercial-size catalysts, the Effectiveness Factor for the commercial-scale catalyst at any point along the catalyst bed can be determined from the Kₒ for the crushed catalyst at that point and the Kᵢ.

For reactions, such as methanol and/or DME to propylene conversion, in which different reaction pathways are possible in different longitudinal portions of the catalyst bed of the fixed bed reactor, e.g., conversion of methanol to higher olefins or to propylene or to carbon, it is important also to characterize the behavior of the different kinetic pathways producing the product and various byproducts that can exist for the system as they vary along the length of the composite catalyst bed of the reactor in order to explore the longitudinally dependent kinetic and mass transfer space for the system, and to distinguish between the occurrence of mass transfer and kinetic effects in the system. When this space has been explored, the mass transfer performance of reactant to product for the system operating at a given set of conditions that involve an optimal set of trade-offs for the particular catalyst can be investigated.

In scaling-up a reactor to commercial size, is preferable to confirm the mass transfer characteristics determined under isothermal conditions in the manner described above in an adiabatic reactor. In an adiabatic reactor, the amount of diluent for the commercial-size catalyst is reduced and the tube diameter is controlled so that its thermal performance mirrors that expected for the commercial-size reactor.

Heat Transfer Effects

Understanding the heat transfer performance of a fixed bed reactor is critical to maximizing the productivity at which the reactor can be run. For methanol and/or DME conversion, the reaction rate is higher at higher temperatures. However, if the temperature is allowed to become too high, there is a danger of temperature runaways. The temperatures in the catalyst bed of a fixed bed reactor can vary both longitudinally and laterally within the catalyst bed. Excess heat is preferably removed through the walls of the reactor to a medium such as circulating boiling water or a fluidized sand bath.

The reactor system illustrated in FIG. 2 of the drawings can also be used to investigate heat transfer characteristics of a methanol and/or DME conversion plug-flow reactor system. For example, the catalyst beds in the reactor stages 13, 15 and 17 of the reactor 11 can contain a mixture of crushed catalyst and inert diluent particles, and the catalyst beds in stages 37, 39 and 41 of the multistage reactor 35 can contain mixtures of full-size catalyst particles and inert diluent particles. And both cases the ratios of catalyst particles to inert diluent particles are selected so that the reactor's 11 and 35 operate substantially isothermally. The catalyst beds of the reactors 11 and 35 are instrumented with thermocouples (not shown) to measure in the temperatures at successive longitudinal positions along the catalyst beds, both in the central portion of the bed cross-section and near its periphery. In addition, the effluent of each of the reactor stages is sampled by sampling valves 25, 27 and 29 of multistage reactor 11 and sampling valves 49, 51 and 53 of multistage reactor 35. Lateral heat transfer effects can be further studied by inserting conductive sleeves in the reactor stages in order to decrease the catalyst bed diameter so that the heat generated in the central portion of the bed has less distance to travel to the heat sink formed by the reactor walls and the temperature control device 33 surrounding the reactor walls. Successively thinner heat conductive sleeves can be used to incrementally increase of the diameter of the catalyst bed until the bed diameter is such that the heat that cannot be adequately removed from the central portion of the bed through the reactor walls.

Temperature and product measurements are preferably repeated for different reactor flow rates, pressures, and productivities, both at Start of Run and during the reactor's time on stream as the reactor lines out. The effect on heat transfer characteristics and other process parameters, such as conversion, selectivity and kinetics, of using catalyst particles of various sizes and shapes in the catalyst bed can also be investigated using the method of the invention. The data obtained from such measurements permits one to investigate and gain an understanding of how the heat transfer properties of the reactor system affect reactor performance over the entire multivariable space in which the commercial-size reactor might operate.

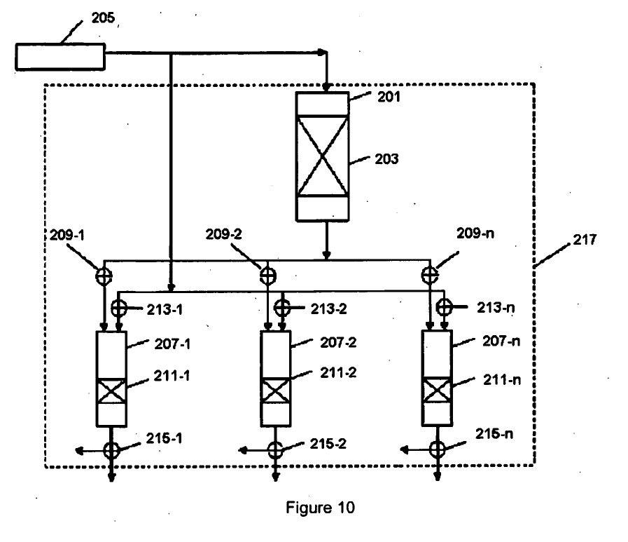

Referring now to FIG. 10 of the drawings, there is illustrated an alternative embodiment of apparatus useful in performing the method of the invention which can be used for investigating the longitudinally dependent mass transfer, kinetics and heat transfer characteristics of a fixed bed reactor. The laboratory scale plug-flow reactor 201 contains a bed 203 of commercial sized catalyst particles. Reactor 201 is supplied with fresh reactant feed from the source 205. Effluent from the reactor 201 is supplied to fixed bed reactor stages 207-1 through 207-n through control valves 209-1 through 209-n for feeding controlled amounts of effluent from reactor 201 to such reactors. Each of the reactor stages 207-1 through 207-n contains a narrow catalyst bed 211-1 through 211-n of catalyst particles mixed with enough inert diluent particles so that the catalyst beds operate in a substantially isothermal mode. The source 205 also supplies controlled amounts of fresh reactant feed to the inlets of the reactor stages 207-1 through 207-n through control valves and 211-1 through 211-n. The effluents from the reactor or stages 207-1 through 207-n can be sampled by means of sampling valves 215-1 through 215-n.

If the reactor 201 is operated at a given conversion level, e.g. 80%, the input to the individual reactor stages 207-1 through 207-n can represent any degree of conversion from zero to 80% by using the control valves 209-1 through 209-n and 213-1 through 213-n to adjust the ratio of reactor 201 effluent to fresh feed being supplied to the individual reactor stages 207-1 through 207-n. Thus, if the valves 209-1 and 213-1 are adjusted such that reactor stage 207-1 receives only effluent from the reactor 201, and the thickness of the catalyst bed 211-1 is such that it performs an additional 5% conversion on such effluent, the catalyst bed 211-1 is equivalent to a cross-sectional slice of a fixed bed reactor in which the conversion between 80 and 85% takes place. Similarly, if the valves 209-2 and 213-2 are adjusted such that the input to reactor stage 207-2 is equivalent to the effluent of a reactor operating at 40% conversion, and the thickness of the catalyst bed 211-2 is such that it performs an additional 5% conversion on such effluent, the catalyst bed to an 11-2 is equivalent to a cross-sectional slice of a catalyst bed in which the conversion between 40 and 45% takes place. Thus, the catalyst beds 211-1 through 211-n can replicate the performance of a cross-sectional slice of a fixed bed reactor positioned at any longitudinal position along the catalyst bed.

The catalyst beds 211-1 through 211-n need not all have the same composition. For instance, the beds 211-1 and 211-2 could contain crushed and commercial-size catalyst particles, respectively, in each case mixed with an amount of inert diluent particles such that the beds operate in isothermal mode. In this case the mass transfer, heat transfer and kinetics characteristics of a cross-sectional slice of a catalyst bed located at any longitudinal position in the catalyst bed can be investigated. In a different application, the catalyst beds 211-1 through 211-n could contain catalyst particles of different chemical or physical composition. In order to prevent heat loss or gain in the effluent from the reactor 201 being fed to the reactor stages 207-1 through 207-n, the connecting tubing and valves are preferably surrounded by insulating material and the entire system comprising the reactor 201 and the reactor stages 207-1 through 207-n can be surrounded by a temperature control device, or alternatively, the reactor 201 and reactor stages 207-1 through 207-n can be surrounded by separate temperature control devices, depending on the needs of the application. Additionally, the reactant feed from the source 205 being supplied to the reactor stages 207-1 through 207-n can be heated before it is supplied to such reactor stages by well-known indirect heating means such as a coil in a sand bath or an infrared furnace (not shown) in order to have the appropriate temperature conditions in the catalyst bed inlet portions of such reactor stages.

The apparatus disclosed in FIGS. 2, 4, 7 and 10 can also be used to investigate other operating parameters of a plug-flow reactor for scale-up or other purposes in accordance with the method of the invention. For example, the longitudinally dependent activity maintenance of a catalyst bed can be investigated as a function of time on stream under different conditions of temperature, pressure and catalyst shape and size. Other longitudinally dependent process parameters that can be investigated using the method of the invention include the effects of different space velocities, reaction products and by-products, different operating temperatures and pressures, time on stream, and different catalyst sizes and shapes, on matters such as e.g., conversion, productivity, kinetics and selectivity, and on changes in catalyst physical and chemical properties such as active site crystal size, oxidation, and growth of an over-layer of support on the surface of the catalyst active sites.

Using present invention, the time for scale-up of the methanol and/or DME to propylene catalytic conversion process from discovery to commercial scale application can be significantly reduced. For example, in one particularly advantageous configuration, four multi-stage reactors of the type described above can be operated in parallel. In this embodiment, the stages of one of the reactors are loaded with crushed catalyst. This reactor provides Intrinsic Reaction Rate and selectivity data. The stages of the second reactor are loaded with commercial-size catalyst. The data from this second reactor can be used to define the degree of mass transfer limitation (Effectiveness Factor) based on a direct comparison of the relative residence times in the reactors containing the crushed catalyst in the commercial-size catalyst required to achieve a given amount of conversion. By obtaining conversion data at a series of residence times, it is possible to determine the Effectiveness Factor and hence the Effective Diffusivity with conversion or residence time. This data also provides information on the impact of mass transfer on selectivity. A third, probe reactor can be operated in parallel with the previous two reactors. This probe reactor can either be a shallow fixed bed reactor or a back-mixed reactor. Flow can be directed to the appropriate actor from any of the reactor beds in the previous two reactors. In addition, additional gases or liquids can be added to the probe reactor to determine the rates of adsorption or surface property changes on the catalyst. This information can provide valuable insight in modeling the fixed bed reactor. Finally, an adiabatic reactor can be operated in parallel to test the reactor model developed from the previous reactors. Operation of the series reactors in this parallel mode allows for much faster generation of the required scale-up data. In fact, all the required scale-up data, including deactivation and regeneration data, at one temperature can be obtained in one to two years, for a savings of several years of development time. A further improvement to the experimental design would be to operate several four reactor sets at the same time. These sets can be operated at different temperature, pressure, and feed compositions. The set producing the optimum economics can be used for the commercial design. The cost of operating several parallel sets of series reactors simultaneously is a small expense when compared to the potential savings associated with accelerating the scale-up of a new catalyst to a full-scale commercial operation.

In an adiabatic reactor, it is possible to produce hot spots in the reactor, which may cause the adiabatic reactor to run away. Also, in an adiabatic reactor, because reaction parameters, such as temperature, kinetics parameters, etc., can change continuously, it is difficult to measure the reaction parameters by direct measurement. Dividing an adiabatic reactor into multistage series-connected reactor stages can help determine reaction parameters at different locations along a flow direction of the reactor, but it is difficult to keep continuities of the reaction parameters, especially temperature, between adjacent reactor stages.

Therefore, it is difficult to directly measure reaction parameters in an adiabatic reactor, and to exactly and securely determine reaction characteristics in the adiabatic reactor, such as kinetics, mass transfer, heat transfer etc.

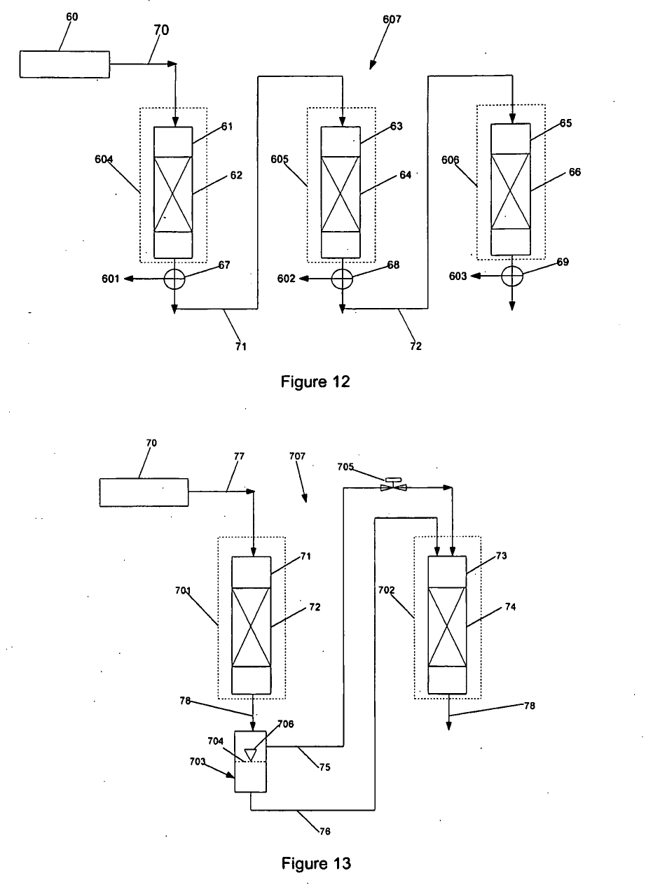

FIG. 12 illustrates a schematic diagram of a composite multistage laboratory scale plug flow reactor 607. The reactor 607 includes first, second and third series-connected reactor stages 61, 63 and 65, each having a catalyst bed 62, 64 and 66. The reactor 607 further includes a fresh reactant conduit 70 which connects an inlet of the first reactor stage 61 to a source 60, so that the source 60 can provide feeds, which are normally fresh reactants, to the first reactor stage 61. The reactor 607 further includes connecting conduits 71 and 72 to connect the first and second reactor stages 61 and 63, and the second and the third reactor stages 63 and 65, respectively. A first sampling valve 67 is disposed between the first and second reactor stages 61 and 63, and has an output 601 to facilitate sampling effluents from the first reactor stage 61. Here in this document, a device is said to be disposed between two stages of the reactor does not necessarily mean that the device is physically disposed between the two stages of the rector but that the device is between the two stages of the reactor along a flow of reactants. A second sampling valve 68 is disposed on the conduit 72 and has an output 602 for sampling effluents from the second reactor stage 63. A third sampling valve 69 is disposed between an outlet of the third reactor stage 65 and a device, such as a fourth reactor stage or a product accumulator (not shown) and has an output 603 for sampling effluents from the third reactor stage 65. A sampling valve connected to the fresh reactant conduit 70 may also be provided in order to permit analysis of the feeds.

In one embodiment, the reactor stages 61, 63 and 65 are isothermal reactor stages, which are used together to simulate an adiabatic reactor. Thus, temperature control devices 604, 605 and 606 are provided to control the temperature of the reactor stages 61, 63 and 65 respectively. A preheater (not shown) may be disposed between the source 60 and the first reactor stage 61 to preheat the feeds from the source 60 so that when the feeds flow into the first reactor stage 61, the feeds have already reached a desired temperature for the feeds. Alternatively, the preheater can also be disposed in the first reactor stage 61.

In one embodiment, when using the isothermal reactor stages 61, 63 and 65 to simulate the characteristics of an adiabatic reactor, the temperature setting for each of the temperature control devices 604, 605 and 606 should be determined first. Generally, for a given catalytic process, based on data derived from operating the adiabatic reactor in practice, the temperature setting for the first temperature control device 604 and temperature variation in the first reactor stage 61 can be determined. Then, based on information from the first reactor stage 61, the temperature setting of the second temperature control device 605 can also be determined, and so on. Thus, after the temperature settings of each of the temperature control devices 604, 605 and 606 is determined, the reactor stages 61, 63 and 65 can be used to simulate the characteristics of the adiabatic reactor.

In this embodiment, the temperature of the temperature control devices 604, 605 and 606 are defined as T1, T2 and T3, which are different from each other. Different catalytic processes may have different T1, T2 and T3 settings. Alternatively, a common temperature control device (not shown) can be provided to control the temperatures of reactor stages 61, 63 and 65 together.

Thus, the isothermal reactor stages 61, 63 and 65 can respectively simulate successive catalyst bed slices of a catalyst bed of a larger adiabatic reactor. Thus, the characteristics of the catalyst bed, which is simulated by the catalyst beds 62, 64 and 66, are determined. Because it is relatively easy to operate the isothermal reactor stages, characteristics associated with the larger adiabatic reactor can be determined by simulating the adiabatic reactor using the isothermal reactor stages. In this embodiment, the first, second and third reactor stages 61, 63 and 65 can be arranged upright.

For a particular catalytic process between at least two successive reactors, for example a particular catalytic process in a multistage series-connected reactor stages, if an effluent fluid from one reactor stage is homogeneous, such as in a gas phase, transferring effluent fluid can be quite straightforward by using a properly sized and shaped tube connecting an outlet of one reactor stage to an inlet of a following reactor stage. In many catalytic processes, however, the effluent from a reactor stage may be in a multiphase state, meaning that it includes one or more gaseous fluids, which are fluids in gas phase (such as gases, vapors or mixtures of gases and vapors), and one or more liquid fluids, which are fluids in one or more liquid phases (such as water phase, oil phase, other immiscible phases and partial emulsion phases, etc.)

The multiphase fluid is often a multi-component fluid, each component being in its own state, which can be a single-phase state or multiphase state. If the multi-component fluid is in thermodynamic equilibrium, the fluid can be transferred directly by a tube connecting two successive reactor stages.

However, in certain catalytic processes, such as hydrodesulphurization etc., the multi-component fluid may not be in thermodynamic equilibrium. So, when the multi-component fluid is transferred directly through the tube connecting the outlet of one reactor stage to the inlet of the following reactor stage, the states of the components may vary during the transfer such that continuity or consistency of the fluid between adjacent two reactor stages may be broken. Thus, it is difficult to use the multistage series-connected reactor stages to model a plug reactor and to measure and optimize the corresponding catalytic processes.

FIG. 13 illustrates a schematic diagram in accordance with one embodiment of the present invention. In this embodiment, a catalytic process development apparatus includes a composite multistage laboratory scale plug flow reactor 707 which includes first and second series-connected reactor stages 71 and 73. The reactor stages 71 and 73 include catalyst beds 72 and 74, respectively. The catalytic process development apparatus further includes temperature control devices 701 and 702 disposed on the reactor stages 71 and 73 respectively, and a fresh reactant conduit 77. The fresh reactant conduit 77 is connected an inlet of the first reactor stage 71 to a source 70 so that the source 70 can provide feeds which are normally fresh reactants to the first reactor stage 71. In this embodiment, the catalytic process development apparatus further includes a separator 703, first and second effluent conduits 78, a gas conduit 75 and a liquid conduit 76. The first conduit 78 is connected an outlet of the first reactor stage 71 to an inlet of the separator 703. The gas conduit 75 and the liquid conduit 76 connect the separator 703 to an inlet of the second reactor stage 73. The second effluent conduit 78 connect an outlet of the second reactor to a following device (not shown), such as another separator. The reactants from the source 70 are fed into the first reactor stage 71. A multiphase effluent fluid from the first reactor stage 71 is sent into the separator 703, wherein gaseous fluid(s) in the multiphase fluid are separated from liquid fluid(s), and both are introduced into the second reactor stage 73 through the gas conduit 75 and the liquid conduit 76 respectively.

Referring to FIG. 13, the catalytic process development apparatus further includes a flow restrictor 705 disposed on the gas conduit 75 to control flow resistance in the gas conduit 75, resulting in a gas pressure difference (pressure drop) ΔP between two sides of the flow restrictor 705. Assuming a gas pressure in the first reactor 71 and the separator 703 is P1, a gas pressure in the second reactor 73 is P2. Thus, P1>P2 due to the flow restrictor 705, and ΔP=P1−P2.

In one embodiment, ΔP is large enough so that it can drive the liquid fluid in the separator 703 to enter into the liquid conduit 76 and to flow into the second reactor stage 73 but is also small enough so that it can not affect reactions in the second reactor stage 73. The flow restrictor 705 can be a restricting valve, an orifice, or other restricting means etc. When properly sized and shaped, the gas conduit 75 can function as the flow restrictor 705. The flow resistance of the gaseous fluid can be adjusted by many ways, such as electrical, electromagnetic, pneumatic, mechanical or thermal ways etc., which are familiar to those ordinary skills in the art. The electromagnetic ways are preferred.

Additionally, the catalytic process development apparatus further includes a differential pressure sensor (not shown) disposed across the flow restrictor 705 or two ends of the gas conduit 75 to measure the ΔP. Combined ΔP and physical properties of the gaseous fluid, information about a mass flow rate of the gaseous fluid can be determined.

In one embodiment, if ΔP is too small, the liquid fluid can not flow but accumulate in the separator 703. If ΔP is too large, the liquid fluid may keep flowing until all the liquid fluid in the separator 703 is transported to the second reactor stage 73. When the liquid fluid in the separator 703 is drawn out, the gaseous fluid may flow through the liquid conduit 76. Thus, ΔP is reduced due to an extra pathway for the gaseous fluid. Then, the liquid fluid begins to accumulate in the separator 703 and blocks the liquid conduit 76. Subsequently, the ΔP restores to a desired value little by little, and the liquid fluid starts to flow again. Thus, the flow rates of the gaseous and liquid fluids may fluctuate with respect to time because of fluctuation of the ΔP, which is disadvantageous to the second reactor stage.

In a preferred embodiment, the catalytic process development apparatus includes a liquid level sensor 706 disposed in the separator 703. The liquid lever sensor 706 monitors variation of a liquid level 704 in the separator 703. Liquid sensor signals from the liquid level sensor 706 are used to control the flow restrictor 705 to generate a suitable ΔP to drive the liquid fluid in such a manner that the liquid level 704 is maintained at a desired substantially constant level. Thus, the fluctuation of the fluids in the separator 703 can be eliminated. When the liquid fluid is transferred stably through the liquid conduit 76, the liquid mass flow rate information can also be obtained by using the measured ΔP in combination with physical properties of the liquid fluid.

In one embodiment, in certain low pressure reactions including low pressure FT synthesis etc., a small pressure drop ΔP may still be too big to tolerate, especially when the reactor stage is long or there are many reactor stages. Additionally, in the process of adjusting ΔP to maintain the liquid level 704 by the liquid level sensor 706 and the flow restrictor 705, the fluctuation of ΔP may also affect liquid flow in the first reactor stage 71.

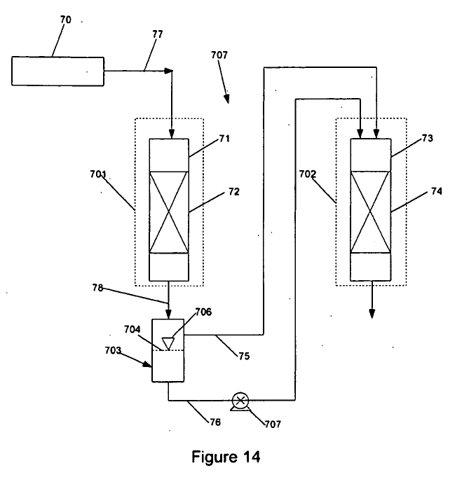

FIG. 14 illustrates a similar schematic diagram as the diagram of FIG. 13. In this embodiment, the flow restrictor 705 is removed from the gas conduit 75, so, there is no pressure drop ΔP on the gaseous fluid. Meanwhile, a liquid pump 707 is disposed on the liquid conduit 76. The liquid level signals are used to control the liquid pump 707 to maintain the liquid level 704 at the desired constant level. Additionally, because an output pressure of the liquid pump 707 is approximately equal to its input pressure, it does not create a pressure drop between the first and the second reactor stages 71 and 73.

In this embodiment, the liquid pump 707 includes a positive displacement pump or a centrifuge pump etc. Additionally, the liquid pump 707 can have metering capability, which can be used to obtain the liquid flow rate information directly. In order to cause the liquid fluid to be distributed uniformly in the second reactor stage 73, a sprayer or similar spraying devices (not shown) can be adopted inside the reactor stage 73. Alternatively, a check valve (not shown) may be disposed on the liquid conduit 76 and located behind the liquid pump 707 to prevent the liquid fluid in the liquid conduit 76 from reflux.

In the embodiments of the present invention, the gaseous fluid and the liquid fluid in the effluent of the first reactor stage 71 are separated in the separator 703, and then transported to the second reactor stage 73. Thus, possible interactions between the gaseous fluid and the liquid fluid in the effluent during transport can be minimized, and the potential of altering the states of the components in the effluent by fluid distribution and recombination processes can be reduced. The continuity or consistency of the components of the fluid can be maintained between the first and second reactor stages 71 and 73. Additionally, separation of the gaseous fluid and the liquid fluid also makes it easy for sampling the fluids for species analysis, whether continuously or intermittently, on-line or off-line.

As mentioned above, in certain catalytic processes, there are different types of liquid phases for the multiphase effluent fluid. In one example of the FT synthesis, its effluent may contain water phase liquid(s) and oil phase liquid(s). In order to transport such multiphase fluid uniformly, an agitation device (not shown) can be provided to cause homogenization of the multiphase fluid. The agitation device may include a mechanical stirring device, a magnetic stirring device or an ultrasonic stirring device etc. In one embodiment, the ultrasonic stirring device is provided, which can be installed near a bottom of the separator 703. The ultrasonic stirring device can provide sufficient homogenization of the liquid fluid, while having minimum interference to the performance of the liquid level sensor 706 and also without significantly increasing liquid temperature.

Referring to FIGS. 13-14, if the separator 703 is operated in a temperature which is higher than that of the first reactor stage 71, portions of volatile species in the liquid phase in the separator 703 may be evaporated and enter into the gas phase so as to alter the states of the species. If the separator 703 is operated in the temperature which is lower than that of the first reactor stage 71, portions of vapors in the gas phase in the separator 703 may be condensed and enter into the liquid phase so as to also alter the states of the species. As a result, variations in the effluent from the first reactor stage 71 can be produced during its transfer to the second reactor stage 73. Therefore, for certain catalytic processes, it is preferred that the temperature of the separator 703 is the same as that of the effluent from the first reactor stage 71. Thus, the states of the species of the effluent are preserved.

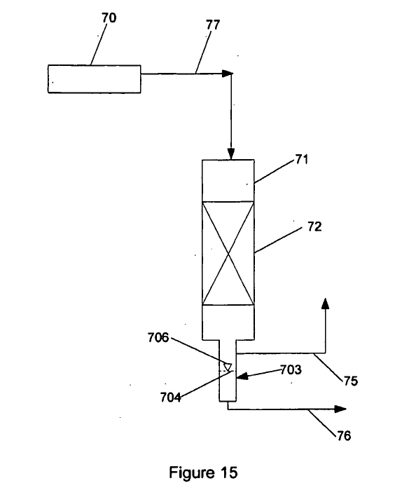

Referring to FIG. 15, for example, in order to keep the temperature of the separator 703 being the same as that of the effluent of the first reactor stage 71, the separator 703 is integrated into the first reactor stage 71. The integrated first reactor stage 71 and the separator 703 can enjoy operation simplicity and also minimize the potential of altering the states of the components.

The composite multistage reactor 707 can include three or more series-connected reactor stages. The outlet of each of the reactor stages can connect to a separator. The separator and the reactor stage can be separate from or integrated with each other. All the reactor stages can also be arranged upright along a vertical line.

1) A method for determining a set of operating parameters for a commercial scale methanol and/or DME to propylene catalytic process and reactor system having a high productivity and selectivity to propylene and a low selectivity for C₅⁺ hydrocarbons, the system including a plurality of series-connected plug-flow reactors, comprising the steps of:
a) simulating said series-connected plug-flow reactors by operating one or more multistage series-connected laboratory scale plug-flow reactors, each of the stages of said laboratory scale reactors containing a catalyst bed that includes a catalyst capable of catalyzing the conversion of methanol or DME to propylene, separate series-connected pluralities of said laboratory scale reactor stages corresponding to separate ones of said series-connected reactors, the simulating step including
i) supplying to the first of the laboratory reactor stages corresponding to the first of said series-connected reactors a fresh feed including selected partial pressures of methanol and/or DME;
ii) supplying to selected ones of said laboratory reactor stages feeds including selected partial pressures of one or more of steam, methanol and/or DME and reaction products, said simulation being performed at a set of operating conditions of temperature, pressure, and reactant and reaction product flow rates, and the catalysts in the catalyst beds of said laboratory scale reactor stages having selected sets of characteristics;
b) repeating the simulating step of step a) at different selected sets of said operating conditions, and/or at different selected sets of characteristics of the catalysts in the catalyst beds of said laboratory scale reactor stages;
c) measuring characteristics and compositions of the effluents of some or all of said laboratory scale reactor stages during each simulating step; and
d) using the results of said measurements obtained in one simulating step to influence the selection of catalyst bed characteristics and operating parameters in subsequent simulating steps for improving the productivity and selectivity of the conversion of methanol and/or DME to propylene by the one or more composite multistage series-connected laboratory scale plug-flow reactors.

2) The method of claim 1 wherein the catalysts in the catalyst beds of said laboratory scale plug-flow reactors include zeolite catalysts.

3) The method of claim 1 wherein the catalysts in the catalyst beds of at least some of the successive stages of the laboratory scale reactors have different acidities and/or reactivities.

4) The method of claim 1 wherein the catalysts in at least some of the catalyst beds of a series-connected plurality of said laboratory scale reactor stages corresponding to one of said series-connected reactors have different acidities and/or reactivities.

5) The method of claim 1 wherein the measuring of characteristics and compositions of effluents of laboratory scale reactor stages during a simulation step includes measurements of some or all of temperature programmed reduction, temperature programmed oxidation techniques, temperature programmed desorption, and surface spectroscopy techniques.

6) The method of claim 1 further including determining the degree of deactivation of the catalyst in the catalyst beds of stages of the laboratory scale reactors during simulating steps.

7) The method of claim 1 wherein the pluralities of laboratory scale reactor stages corresponding to successive ones of said series-connected reactors are connected in series to each other.

8) The method of claim 6 further including regenerating the catalyst in catalyst beds of stages of the laboratory scale reactors to restore some or all of its original activity by treating such catalysts with hydrogen, oxygen, steam or an organic solvent a mixture of selected ones thereof at selected regeneration conditions, and measuring the degree of regeneration.

9) The method of claim 8 wherein said regenerating conditions include treatment with an oxygen or hydrogen containing gas at temperatures ranging from 200 to 700 degree C. at pressures from 1 to 100 bar for a period sufficient to restore at least some of its original activity.

10) The method of claim 1 further including
a) providing a probe reactor operating in parallel with one or more selected series-connected stages of one of said multistage laboratory scale reactors;
b) feeding a portion of the feed to the first of said one or more series-connected stages to the input of said probe reactor;
c) simultaneously feeding another input gas to the input of said probe reactor; and
d) measuring the effect of said other input gas on the catalytic reaction by comparing the effluents of the last of said one or more selected series connected stages with the effluent of the probe reactor.

11) A method for developing a set of operating parameters for a catalyst regeneration process for a commercial-scale methanol and/or DME to propylene plug-flow catalytic process and reactor system, comprising the steps of:
a) partially deactivating a methanol and/or DME to propylene catalyst by operating a methanol and/or DME to propylene catalytic plug-flow process in a composite multistage series-connected laboratory scale plug-flow reactor;
b) during the operation of said methanol and/or DME to propylene process, determining the extent of deactivation of the catalyst beds in reactor stages of said multistage laboratory scale reactor by measuring the relative concentrations of at least some of methanol, DME, propylene, CO, CO₂, H₂O and hydrocarbons in the effluents of the reactor stages of said multistage laboratory scale reactor while operating said methanol and/or DME to propylene process;
c) determining the nature of deactivating chemical and physical changes in the catalyst beds;
d) after a selected degree of deactivation has occurred in the catalyst beds in one or more of said stages, supplying a regenerating gas to said catalyst beds at regenerating conditions for regenerating said catalyst beds, said gas including one or more of hydrogen, oxygen, steam, or an organic solvent;
e) thereafter exposing said catalyst beds to methanol and/or DME to propylene catalytic process operating conditions and measuring the relative concentrations of at least some of methanol, DME, propylene, CO, CO₂, H₂O and hydrocarbons in the effluents of said catalyst beds to determine the extent to which said catalyst beds have been regenerated; and
f) repeating steps b through e using different selected sets of regenerating gas and/or regenerating conditions.

12) The method of claim 11 wherein said step of determining the nature of deactivating chemical and physical changes includes sampling the catalyst particles in stages of said laboratory scale reactor.

13) The method of claim 11 wherein the said commercial scale plug-flow system includes a plurality of series-connected plug-flow reactors and wherein said deactivating step includes operating a methanol and/or DME to propylene catalytic plug-flow process in a plurality of composite multistage series-connected laboratory scale plug-flow reactors, each of said laboratory scale reactors corresponding to a different one of the series connected plug-flow reactors of said commercial scale plug-flow system.

14) A method for developing a set of operating parameters for a catalyst regeneration process for a commercial-scale methanol and/or DME to propylene plug-flow catalytic process and reactor system, comprising the steps of:
a) operating a methanol and/or DME to propylene catalytic plug-flow process in a composite multistage series-connected laboratory scale plug-flow reactor;
b) diverting a portion of the effluent of a stage of said laboratory scale plug-flow reactor to the input of a methanol and/or DME to propylene probe reactor;
c) selectively adding olelfins, dienes, acetylenes, aromatics or other unsaturates to the input of said probe reactor;
d) measuring and comparing the degree of deactivation over time in the probe reactor and in the stage of said laboratory scale reactor following the stage whose effluent was partially diverted to said probe reactor;
e) determining and comparing the degree of reversibility of the deactivation in the probe reactor and in said following stage of said laboratory scale reactor including by supplying a regenerating gas to said probe reactor and said following stage at regenerating conditions and thereafter operating said laboratory scale and probe reactors under methanol and/or DME to propylene catalytic process operating conditions and measuring characteristics and compositions of the effluents of said probe reactor and said following stage; and
f) repeating steps (a) through (e) using different selected sets of regenerating gas and/or regenerating conditions for developing a set of regeneration operating parameters for the commercial scale reactor.

15) The method of claim 14 wherein step (d) includes subjecting the catalysts from said probe reactor and said following reactor stage to surface or bulk analytical characterization to determine the nature of chemical or physical property changes that occurred.

16) The method of claim 14 wherein step (e) includes subjecting the catalysts from said probe reactor and following reactor stage to surface or bulk analytical characterization to determine the nature of chemical or physical property changes that occurred.
